(19) United States
(12) Patent Application Publication
(10) Pub. No.: US 2012/0107916 A1
(43) Pub. Date: May 3, 2012

(54) METHODS AND COMPOSITIONS FOR AFFECTING THE DIFFERENTIATION OF CLOSTRIDIA IN CULTURE

(76) Inventor: Donald Mattsson, Duluth, MN (US)

(21) Appl. No.: 13/379,390

(22) PCT Filed: Jun. 29, 2010
(86) PCT No.: PCT/US2010/040301

§ 371 Date: Dec. 20, 2011

Related U.S. Application Data
(60) Provisional application No. 61/221,996, filed on Jun. 30, 2009.

(51) Int. Cl. C12N 1/20; C07K 2/00

(52) U.S. Cl. 435/252.7; 530/300

(57) ABSTRACT

The invention relates generally to methods and compositions for maintaining and manipulating microbial cultures of Gram-positive bacteria. Also provided are methods for identifying quorum sensing regulatory proteins and auto-inducing peptides in Gram-positive bacteria. Also provided are methods and compositions for affecting quorum sensing pathways of the genus Clostridium in culture including auto-inducing peptides to direct or maintain Clostridium cultures in a desired differentiated state. Differentiated states include extended serial propagation for the production of butanol or other fermentation products.

This patent application claims benefit of priority to U.S. provisional patent application Ser. No. 61/221,996, filed Jun. 30, 2009, incorporated herein by reference in its entirety.

The instant application contains a lengthy Sequence Listing which has been submitted via text file, Annex C/ST.25.txt (.txt), in lieu of a printed paper (or .pdf) copy, and is hereby incorporated by reference in its entirety.

### FIELD OF THE INVENTION

The invention relates generally to methods and compositions for maintaining and manipulating microbial cultures of Gram-positive bacteria. Specifically the invention relates to methods and compositions for affecting quorum sensing pathways of the genus Clostridium in culture to direct or maintain Clostridia cultures in a desired differentiated state.

### BACKGROUND

The growth of the biofuels industry has been driven largely by increases in oil prices, which are not likely to decline in the coming years. Butanol, produced by fermentation, has attractive features as a biofuel such as higher energy content and lower volatility than ethanol. Butanol can also be used as a feedstock chemical for the chemical industry, replacing oil, while ethanol cannot. The production of acetone and butanol using Clostridium acetobutylicum was one of the first large-scale industrial fermentation processes ever developed. Subsequently, Clostridium beijerinckii and other species of solvent-producing Clostridia were used in commercial applications around the world. With increased oil production and lower oil prices from the 1950s and onward innovation in the biobutanol industry has waned.

The use of Clostridium to produce butanol or other solvents may be greatly improved if the various stages of culture could be controlled. When cultured in batch culture, growth of the solvent-producing Clostridia is initially exponential, with the production of acetate, butyrate, carbon dioxide, and hydrogen. As the culture progresses, the pH of the media drops, followed by slowed growth and the production of acetone, butanol, and ethanol. The metabolic shift from acid to solvent production is accomplished by genetic repression of acidogenic enzyme genes and induction of solventogenic enzyme genes. These changes are beneficial for butanol production and advantageous for the biofuels industry. However, many solvent-producing Clostridia lose the ability to produce solvents after repeated subculturing. This phenomenon known as degeneration reduces the usefulness of solvent producing Clostridia. There exists a long felt need to control the various differentiated states of Clostridia in culture, to establish and maintain continuous cultures of Clostridia, and to be able to establish repeated batch cultures while maintaining the capacity for solventogenesis. This ability would reduce degeneration in cultured Clostridia and enhance the usefulness of this organism for industrial applications such as the production of butanol.

### SUMMARY

One embodiment relates to autoinducing peptides which may be used to direct or maintain Clostridium in a desired differentiated state in culture.

Another embodiment relates to methods of using autoinducing peptides to modify the activity of quorum sensing regulatory proteins, to direct or maintain Clostridium in a desired differentiated state in culture.

In yet another embodiment relates to autoinducing peptides and methods used to extend serial propagation of Clostridium in culture.

Another embodiment relates to quorum sensing regulatory proteins, and methods and composition for modifying their activity to direct or maintain Clostridium in a desired differentiated state in culture.

In yet another embodiment, are methods for identifying autoinducing peptides and quorum sensing regulatory proteins in gram positive bacteria.

### DESCRIPTION OF THE FIGURES

FIG. 1 shows stationary phase growth measurements of Clostridium acetobutylicum ATCC 824 batch cultures during sequential transfers in YEPG medium. Spore stocks were germinated and grown anaerobically overnight at 30° C. before beginning sequential transfer every 24 hours of 75 μL of culture to 10 mL fresh YEPG. Cultures were grown for 96 hours after transfer before taking measurements. After germination the cultures were either not treated ( [symbol] ) or were treated with 1 nM ( [symbol] ), 10 nM ( [symbol] ) or 50 nM ( [symbol] ) of Peptide SEQ ID NO:143.

FIG. 2 shows pH measurements of stationary phase C. acetobutylicum ATCC 824 batch cultures during sequential transfers in YEPG medium. Spore stocks were germinated and grown anaerobically overnight at 30° C. before beginning sequential transfer every 24 hours of 75 μL of culture to 10 mL fresh YEPG. Cultures were grown for 96 hours after transfer before taking measurements. After germination the cultures were either not treated ( [symbol] ) or were treated with 1 nM ( [symbol] ), 10 nM ( [symbol] ) or 50 nM ( [symbol] ) of Peptide SEQ ID NO:143.

FIG. 3 shows ceric ion reactive compounds in stationary phase broths of C. acetobutylicum ATCC 824 batch cultures during sequential transfers in YEPG medium. Spore stocks were germinated and grown anaerobically overnight at 30° C. before beginning sequential transfer every 24 hours of 75 μL of culture to 10 mL fresh YEPG. Cultures were grown for 96 hours after transfer before taking measurements. After germination the cultures were either not treated ( [symbol] ) or were treated with 1 nM ( [symbol] ) 10 nM ( [symbol] ) or 50 nM ( [symbol] ) of Peptide SEQ ID NO:143.

FIG. 4 shows stationary phase growth measurements of C. beijerinckii NCIMB 8052 batch cultures during sequential transfers in YEPG medium. Spore stocks were germinated and grown anaerobically overnight at 30° C. before beginning sequential transfer every 24 hours of 75 μL of culture to 10 mL fresh YEPG. Cultures were grown for 96 hours after transfer before taking measurements. After germination the cultures were either not treated ( [symbol] ) or were treated with 1 nM ( [symbol] ), 10 nM ( [symbol] ) or 50 nM ( [symbol] ) of Peptide SEQ ID NO:145.

FIG. 5 shows pH measurements of stationary phase C. beijerinckii NCIMB 8052 batch cultures during sequential transfers in YEPG medium. Spore stocks were germinated and grown anaerobically overnight at 30° C. before beginning sequential transfer every 24 hours of 75 μL of culture to 10 mL fresh YEPG. Cultures were grown for 96 hours after transfer before taking measurements. After germination the cultures were either not treated ( [symbol] ) or were treated with 1 nM ( [symbol] ), 10 nM ( [symbol] ) or 50 nM ( [symbol] ) of Peptide SEQ ID NO:145.

FIG. 6 shows ceric ion reactive compounds in stationary phase broths of C. beijerinckii NCIMB 8052 batch cultures during sequential transfers in YEPG medium. Spore stocks were germinated and grown anaerobically overnight at 30° C. before beginning sequential transfer every 24 hours of 75 μL of culture to 10 mL fresh YEPG. Cultures were grown for 96 hours after transfer before taking measurements. After germination the cultures were either not treated ( [symbol] ) or were treated with 1 nM ( [symbol] ), 10 nM ( [symbol] ) or 50 nM ( [symbol] ) of Peptide SEQ ID NO:145.

FIG. 7 shows stationary phase growth measurements of C. acetobutylicum ATCC 824 batch cultures grown at 37° C. during sequential transfers in YEPG medium. Spore stocks were germinated in the absence of ( [symbol] ) and presence of ( [symbol] ) 50 nM Peptide SEQ ID NO:143. Germinating cultures were grown anaerobically overnight at 37° C. before beginning sequential transfer every 24 hours of 10 μL of culture to 10 mL fresh YEPG. The culture germinated in the presence of added peptide was transferred only to fresh medium that contained added peptide ( [symbol] ). The culture germinated without added peptide was transferred to fresh medium without added peptide ( [symbol] ), and to fresh medium that contained added peptide ( [symbol] ). Cultures were grown for 72 hours after transfer before taking measurements.

FIG. 8 shows pH measurements of stationary phase C. acetobutylicum ATCC 824 batch cultures grown at 37° C. during sequential transfers in YEPG medium. Spore stocks were germinated in the absence of ( [symbol] ) and presence of ( [symbol] ) 50 nM Peptide SEQ ID NO:143. Germinating cultures were grown anaerobically overnight at 37° C. before beginning sequential transfer every 24 hours of 10 μL of culture to 10 mL fresh YEPG. The culture germinated in the presence of added peptide was transferred only to fresh medium that contained added peptide ( [symbol] ). The culture germinated without added peptide was transferred to fresh medium without added peptide ( [symbol] ), and to fresh medium that contained added peptide ( [symbol] ). Cultures were grown for 72 hours after transfer before taking measurements.

FIG. 9 shows ceric ion reactive compounds in stationary phase broths of C. acetobutylicum ATCC 824 batch cultures grown at 37° C. during sequential transfers in YEPG medium. Spore stocks were germinated in the absence of ( [symbol] ) and presence of ( [symbol] ) 50 nM Peptide SEQ ID NO:143. Germinated cultures were grown anaerobically overnight at 37° C. before beginning sequential transfer every 24 hours of 10 μL of culture to 10 mL fresh YEPG. The culture germinated in the presence of added peptide was transferred only to fresh medium that contained added peptide ( [symbol] ). The culture germinated without added peptide was transferred to fresh medium without added peptide ( [symbol] ), and to fresh medium that contained added peptide ( [symbol] ). Cultures were grown for 72 hours after transfer before taking measurements.

FIG. 10 shows stationary phase growth measurements of C. beijerinckii NCIMB 8052 batch cultures grown at 37° C. during sequential transfers in YEPG medium. Spore stocks were germinated in the absence of ( [symbol] ) and presence of ( [symbol] ) 50 nM Peptide SEQ ID NO:145. Germinating cultures were grown anaerobically overnight at 37° C. before beginning sequential transfer every 24 hours of 10 μL of culture to 10 mL fresh YEPG. The culture germinated in the presence of added peptide was transferred only to fresh medium that contained added peptide ( [symbol] ). The culture germinated without added peptide was transferred to fresh medium without added peptide ( [symbol] ), and to fresh medium that contained added peptide ( [symbol] ). Cultures were grown for 72 hours after transfer before taking measurements

FIG. 11 shows pH measurements of stationary phase C. beijerinckii NCIMB 8052 batch cultures grown at 37° C. during sequential transfers in YEPG medium. Spore stocks were germinated in the absence of ( [symbol] ) and presence of ( [symbol] ) 50 nM Peptide SEQ ID NO:145. Germinating cultures were grown anaerobically overnight at 37° C. before beginning sequential transfer every 24 hours of 10 μL of culture to 10 mL fresh YEPG. The culture germinated in the presence of added peptide was transferred only to fresh medium that contained added peptide ( [symbol] ). The culture germinated without added peptide was transferred to fresh medium without added peptide ( [symbol] ), and to fresh medium that contained added peptide ( [symbol] ). Cultures were grown for 72 hours after transfer before taking measurements.

FIG. 12 shows ceric ion reactive compounds in stationary phase broths of C. beijerinckii NCIMB 8052 batch cultures grown at 37° C. during sequential transfers in YEPG medium. Spore stocks were germinated in the absence of ( [symbol] ) and presence of ( [symbol] ) 50 nM Peptide SEQ ID NO:145. Germinating cultures were grown anaerobically overnight at 37° C. before beginning sequential transfer every 24 hours of 10 μL of culture to 10 mL fresh YEPG. The culture germinated in the presence of added peptide was transferred only to fresh medium that contained added peptide ( [symbol] ). The culture germinated without added peptide was transferred to fresh medium without added peptide ( [symbol] ), and to fresh medium that contained added peptide ( [symbol] ). Cultures were grown for 72 hours after transfer before taking measurements.

### DETAILED DESCRIPTION

Disclosed are methods and compositions to manipulate or modify organisms of the genus Clostridium in culture. Specifically disclosed are methods and compositions directed at reducing or delaying the degeneration of a Clostridium culture, whereby the culture stops producing solvents and produces only organic acids. More specifically, these methods and compositions are aimed at directing Clostridium organisms towards a particular differentiated state, or for enhancing or diminishing a particular differentiated state of Clostridium organisms in culture. Such differentiated states include but are not limited to exponential growth, solventogenesis, acidogenesis, granulose synthesis, extended serial propagation or the ability of cells to propagate solventogenic cultures serially, and sporogenesis.

Clostridium cultures are typically initiated from spores under anaerobic conditions. They are allowed to grow in exponential growth phase where they produce acetic and butyric acids and eventually shift their metabolism to solvent production. The metabolic shift typically corresponds to a pH of about 4.8 or lower, depending on the species. Clostridium cultures may also be initiated with active organisms instead of spores. The use of active organisms is preferable because it eliminates the germination stage and allows the culture to enter the exponential growth phase rapidly. The use of active cultures suffers from a significant limitation where after inoculation of 2 to 3 sequential batch cultures or the equivalent number of generations in continuous culture the culture degenerates, in that it stops producing butanol or other solvents and returns to producing only organic acids.

A method of manipulating or modifying the various stages of differentiated Clostridium culture is highly desirable. For example, it may be desirable to begin exponential growth earlier to increase the initial number of organisms in the culture. It may be desirable to begin solventogenesis earlier and maintain it longer to maximize the fermentation of butanol or other solvents. It may also be desirable at times to initiate granulose synthesis and generate granulose storage cells or clostridial from cells. The ability to extend sequential batch cultures or continuous cultures using inoculums of active cultures instead of spores, with the cultures being fully capable of butanol production is highly desirable for efficient and economic butanol production. In addition, the ability to generate spores is desirable for intermediate or long term storage of Clostridium organisms. Particularly, it is highly desirable to avoid culture degeneration and to be able to extend sequential batch cultures or continuous cultures from active cultures while maintaining the ability to produce butanol. The molecular mechanisms underlying the shift towards one differentiated state or another, or towards culture degeneration are not known. However, a long felt need exists for a method of directing or maintaining differentiation in Clostridium cultures.

Observations of synchronous behavior of Clostridium organisms in culture suggested to the Inventor that quorum sensing mechanisms may be operating. Quorum sensing is a mechanism by which populations of bacteria coordinate some aspect of their behavior according to the local density of their population. For example, in Bacillus, gene expression can be regulated according to population density by recognition of oligopeptide autoinducing peptides in the culture media that directly bind to effector proteins in responding cells (Bongiorni, et al., (2005), J. of Bacteriology, 187: 4353-4361). No such system is known in Clostridium. However the Inventor reasoned that a similar system, if present in Clostridium, may be manipulated to induce or maintain the various differentiated stages of culture, including but not limited to exponential growth, solventogenesis, acidogenesis, granulose synthesis, extended serial propagation, and sporogenesis. In one embodiment, a peptide with a sequence corresponding to an autoinducing peptide is added to the culture medium of a Clostridium culture in sufficient amount to affect quorum sensing regulatory proteins in responding cells, and thereby directs or maintains the culture in a desired differentiated state. By providing an effective amount of autoinducing peptide or peptides, the various differentiated states may be initiated or maintained.

To manipulate or modify Clostridium cultures in the described manner it is first necessary to identify specific autoinducing peptides and/or their quorum sensing regulatory proteins. Although quorum sensing pathways are known in other bacterial genera, it is difficult or impossible to predict which, if any quorum sensing pathway may be active in another bacterial genus or which regulatory function may be assigned, and which if any autoinducing peptide will activate or deactivate that pathway.

### I. Quorum Sensing Regulatory Pathways

The first step in the discovery of quorum sensing pathways in Clostridium was to indentify quorum sensing regulatory proteins. Although quorum sensing regulatory proteins are not known in Clostridium, it was reasoned that a putative quorum sensing regulatory protein may share conserved sequences with quorum sensing regulatory proteins of other species. For example, PlcR is a virulence regulator of Bacillus cereus (see Declerck et al., (2007), Proc. Natl. Acad. Sci., 104:18490-18495). PapR is an autoinducing peptide that promotes virulence in B. cereus. PapR is secreted by B. cereus and then imported back into the cell across the cell membrane. Increased bacterial densities result in increased PapR concentrations in the media and inside the bacteria, thereby allowing increased interaction of PapR with PlcR. A PapR:PlcR complex is formed, which binds to a specific DNA recognition site, a palindromic PlcR box, that activates a positive feedback loop to up-regulate the expression of PlcR, PapR, as well as various other B. cereus virulence factors. The PapR gene is located 70 by down stream from PlcR. It encodes a 48 amino acid peptide which is secreted, then imported back into the bacteria by an oligopermease in the cell membrane. It is thought that once internalized, PapR undergoes further processing and that a heptapeptide derived from PapR interacts with PlcR, which allows binding to its DNA target thereby activating PlcR regulatory mechanisms. The PlcR protein is known to contain 11 helices, which form five tetratricopeptide repeats (TPR). The structure of PlcR is also similar to the structure of PrgX, an autoinducing peptide of another gram-positive bacteria Enterococcus faecalis. However, PlcR and PrgX control different processes in these different bacterial genera. PlcR, PrgX, the Bacillus thuringiensis NprR protein, and the Rap family of proteins in Bacillus, all possess TPR units. These proteins belong to a superfamily of proteins known as RNPP for Rap/NprR/PlcR/PrgX. Despite structural similarities within this superfamily it is not possible to predict which if any function may be attributed to a particular quorum sensing regulatory protein pathway or which if any autoinducing peptides may activate that pathway.

It was reasoned that if regulatory sequences were present in Clostridium they may possess tetratricopeptide repeats or share homology to PlcR and other members of the RNPP superfamily. In addition, since genes for autoinducing peptides may share genetic regulation factors with genes for their quorum sensing regulatory protein targets, they may be located in close proximity in the genome and possibly downstream from the regulatory protein genes. It was also reasoned that since quorum sensing autoinducing peptides require export from the bacterium, they may be associated with polypeptide secretory sequence signals. Finally, since an active autoinducing peptide sequence may be the result of proteolytic modification of the gene product, the actions of proteases on the putative sequences were considered.

PlcR and PrgX as well as other members of the RNPP family were used to search for homologs among predicted protein sequences in genomic sequence data for solventogenic Clostridia using PSI Blast. Using this approach 46 suspected quorum sensing regulatory protein sequences were identified in C. acetobutylicum ATCC 824 (Table 2) and 28 in C. beijerinckii NCIMB 8052 (Table 3). When regions downstream from suspected quorum sensing regulatory protein sequences were examined for encoded polypeptides, 33 were identified in C. acetobutylicum ATCC 824 (Table 5) and 19 in C. beijerinckii NCIMB 8052 (Table 6). When examining these sequences for putative autoinducing peptides associated with secretory signals, 4 peptides in C. acetobutylicum ATCC 824 and 1 peptide in C. beijerinckii NCIMB 8052 were identified (Table 7). From these 5 sequences, 3 possessed attributes present in other quorum sensing systems. These 3 sequences were used to further search against the genomes of C. acetobutylicum and C. beijerinckii, and 2 additional sequences were identified (Table 8). Utilizing this strategy has lead to the discovered of a new class of quorum sensing regulatory pathways, quorum sensing regulatory proteins, and autoinducing peptides belonging to the genus Clostridium. These quorum sensing regulatory proteins and/or their respective autoinducing peptides may be manipulated or modified to control events such as exponential growth, solventogenesis, acidogenesis, granulose synthesis, extended serial propagation, and sporogenesis.

The modification of any component of a quorum sensing regulatory pathway may direct or maintain a culture of Clostridium organisms in a desired differentiated state. One non-limiting example includes the use of autoinducing peptides in the Clostridium culture media. In addition to the use of autoinducing peptides, other non-limiting examples include altering or modifying the transcription, translation, or post-translational modification of quorum sensing regulatory proteins, oligopermeases, or autoinducing peptides. The modification through genetic engineering or other means of any quorum sensing pathway component may result, for example, in changes to the export or uptake of autoinducing peptides, the interaction of autoinducing peptides with either quorum sensing regulatory proteins, oligopermeases, or other relevant components, and successfully manipulate or modify the behavior of Clostridium organisms in culture.

In one embodiment, an effective amount of autoinducing peptide or peptides may be added singly or in combination, initially or continuously, to the culture medium of a Clostridium culture, at any stage of cell culture, to maintain or achieve a desired differentiated state. Any stage of culture includes but is not limited to: inoculation; growth phase including, lag, exponential, and stationary phases; death phase; acidogenic phase; solventogenic phase; sporogenesis phase; just prior to removal of organisms for inoculation of a subsequent batch or continuous culture; and a time just after signs of culture degeneration are detected.

In one preferred embodiment, an effective amount of autoinducing peptide or peptides are added to the media of a culture of a butanol producing strain of Clostridium at inoculation or during culture to maintain or increase the degree and duration of solvent formation during batch, sequential batch, fed-batch or semi-continuous culture, or continuous culture. Non-limiting examples of preferred autoinducing peptides are set forth in SEQ ID NO: 143, SEQ ID NO: 144, SEQ ID NO: 145, SEQ ID NO: 146, SEQ ID NO: 147 and SEQ ID NO: 148.

In another embodiment, an effective amount of autoinducing peptide or peptides are added to the media of a culture of a butanol producing strain of Clostridium at inoculation or during culture to extend serial propagation of the culture and maintain or increase the degree and duration of solvent formation during batch, sequential batch, fed-batch or semi-continuous culture, or continuous culture. Non-limiting examples of preferred autoinducing peptides are set forth in SEQ ID NO: 143, SEQ ID NO: 144, SEQ ID NO: 145, SEQ ID NO: 146, SEQ ID NO: 147 and SEQ ID NO: 148.

In another embodiment, an effective amount of autoinducing peptide or peptides as set forth in SEQ ID NO: 143, SEQ ID NO: 144, SEQ ID NO: 146, and SEQ ID NO: 148 is added to the media of Clostridium acetobutylicum during culture to maintain or increase the degree and duration of solvent formation during batch, sequential batch, fed-batch or semi-continuous culture, or continuous culture.

In another embodiment, an effective amount of autoinducing peptide or peptide as set forth in SEQ ID NO: 145, and SEQ ID NO: 147 is added to the media of Clostridium beijerinckii during culture to maintain or increase the degree and duration of solvent formation during batch, sequential batch, fed-batch or semi-continuous culture, or continuous culture.

In yet another embodiment, the genetic regulation of autoinducing peptide production by the Clostridia may be genetically engineered whereby the autoinducing peptide is increased or decreased, thereby providing elevated or diminished levels of autoinducing peptides in the culture media. Alternatively, any cell capable of co-culture with Clostridium may be genetically engineered to secrete an autoinducing peptide into the culture media thereby providing a source of autoinducing peptide or peptides.

In yet another embodiment, the quorum sensing regulatory protein may be altered to activate or deactivate the quorum sensing pathway. By way of example, a genetically engineered Clostridium organism may possess a quorum sensing regulatory protein that performs its translational regulatory function without the requirement of binding an autoinducer peptide. Non-limiting examples of quorum sensing regulatory proteins are set forth in SEQ ID NO: 17 through SEQ ID NO:142.

In yet another embodiment, the expression or function of a quorum sensing regulatory protein is reduced or eliminated in order to direct or maintain an organism in a desired differentiated state. By way of example, a quorum sensing regulatory protein that has an inhibitory effect on extended serial propagation is reduced or eliminated using genetic engineering methods to produce what is commonly known as a knock-out organism. Such an organism lacking the inhibitory regulatory function may be directed to or maintained in a state of extended serial propagation. Non-limiting examples of inhibitory regulatory proteins include SEQ ID NO: 26 and SEQ ID NO: 145.In yet another embodiment the oligopermeases of a quorum sensing regulatory pathway may be altered to increase or decrease the amount of autoinducing peptide inside the bacterium. By way of example a genetically engineered Clostridium organism with increased numbers of oligopermeases may result in increased import of specific autoinducing peptides into the bacterium thereby activating greater numbers of quorum sensing regulatory proteins resulting in an elevated cellular response.

In yet another embodiment is a method of identifying quorum sensing regulatory proteins in Clostridium organisms by searching a Clostridium genome, and identifying encoded polypeptides with TPRs, or homology with RNPP proteins. Non-limiting examples of Clostridium genomes are set forth in SEQ ID NO:14, SEQ ID NO:15 and SEQ ID NO:16. Non-limiting examples of RNPP proteins are set forth in SEQ ID NO:1 through SEQ ID NO:13.

In yet another embodiment is a method of identifying autoinducing peptides in Clostridium by searching a Clostridium genome and identifying polypeptides in close linear proximity to quorum sensing regulatory proteins and also close linear proximity to Clostridium secretory signal proteins.

In yet another embodiment is a method of identifying autoinducing peptides in any Gram positive bacteria by searching a Gram positive bacteria genome and identifying polypeptides in close linear proximity to quorum sensing regulatory proteins and also close linear proximity to Gram positive bacteria secretory signal proteins.

The aforementioned alterations or genetic modifications are well known in the art and may include any number of changes in, for example, gene regulatory regions, or protein coding regions, including insertions, deletions, frame shift mutations and point mutations, alteration of stop codons and knock-out mutations. These elements of the inventors' methodology are generally well known and described in detail in numerous laboratory protocols, two of which are Molecular Cloning 2nd edition, (1989), Sambrook, J., Fritsch, E. F. and Maniatis, J., Cold Spring Harbor, and Molecular Cloning 3rd edition, (2001), J. F. Sambrook and D. W. Russell, ed., Cold Spring Harbor University Press, incorporated herein in their entirety by reference. Any number of methods known in the art may be used to accomplish the genetic alterations or modifications in Clostridium. One example includes a method that uses a genetic vector that is based on a modified Group II introns. In particular, the Lactococcus lactis L1.LtrB Group II intron as described in WO 2007/148091, and incorporated herein by reference in its entirety. The method allows targeted, stable disruption of any gene for which the sequence is known by incorporating a specific target sequence into the vector, which also contains a selectable marker. Following genetic transformation of cells the vector integrates into the targeted gene, based on the target sequence, and integrants are selected by virtue of the selectable marker. Finally, the selectable marker is excised from the integrated vector by the activity of a specific recombinase enzyme and the selectable phenotype is lost, while the remainder of the vector remains in the targeted integration site disrupting the targeted gene. In more detail, the vector contains a modified Group II intron which does not express the intron-encoded reverse transcriptase but which does contain a modified selectable marker gene in the reverse orientation relative to the modified Group II intron, wherein the selectable marker gene comprises a region encoding a selectable marker and a promoter operably linked to said region, which promoter is capable of causing expression of the selectable marker encoded by a single copy of the selectable marker gene in an amount sufficient for the selectable marker to alter the phenotype of a bacterial cell of the class Clostridia such that it can be distinguished from the bacterial cell of the class. Clostridia lacking the selectable marker gene; and a promoter for transcription of the modified Group II intron, said promoter being operably linked to said modified Group II intron; and wherein the modified selectable marker gene contains a Group I intron positioned in the forward orientation relative to the modified Group II intron so as to disrupt expression of the selectable marker; and wherein the DNA molecule allows for removal of the Group I intron from the RNA transcript of the modified Group II intron to leave a region encoding the selectable marker and allows for insertion of said RNA transcript (or a DNA copy thereof) at a site in a DNA molecule in a bacterial cell of the class Clostridia. One example of a selectable marker may be a gene for a particular antibiotic resistance, thus selection is accomplished by exposing the cells in culture to the particular antibiotic. The modified Group II intron described above can also contain specific targeting portion, which allow for the insertion of the RNA transcript of the modified Group II intron into a site within a DNA molecule in the clostridial cell. Typically, the site is a selected site, and the targeting portions of the modified Group II intron are chosen to target the selected site. Non-limiting examples of target sites may be quorum sensing regulatory proteins or autoinducing peptides. Preferably, the selected site is in the chromosomal DNA of the Clostridial cell. Typically, the selected site is within a particular gene, or within a portion of DNA which affects the expression of a particular gene, or within a portion of DNA which affects the expression of a particular gene. Insertion of the modified Group II intron at such a site typically disrupts the expression of the gene and leads to a change in phenotype. By way of example, if the quorum sensing regulatory protein is inhibiting extended serial propagation, the inhibition would be removed, and the phenotype would change towards extended serial propagation. Other examples of target sites include autoinducing peptides which may be modified by the insertion of alternative promoters or multiple copies of genes for the autoinducing peptides which result in production or increased production of the particular autoinducing peptide. The selectable marker gene or its coding region may be associated with regions of DNA for example flanked by regions of DNA that allow for the excision of the selectable marker gene or its coding region following its incorporation into the chromosome. Thus, a clone of a mutant Clostridial cell expressing the selectable marker is selected and manipulated to allow for removal of the selectable marker gene. Recombinases may be used to excise the region of DNA. Recombinases may be endogenous or exogenous. Typically, recombinases recognize particular DNA sequences flanking the region that is excised. Cre recombinase or FLP recombinase are preferred recombinases. Alternatively, an extremely rare-cutting restriction enzyme could be used, to cut the DNA molecule at restriction sites introduced flanking the selectable marker gene or its region. A mutant bacterial cell from which the selectable marker gene has been excised retains the Group II intron insertion. Accordingly, it has the same phenotype due to the insertion with or without the selectable marker gene. Such a mutant bacterial cell can be subjected to a further mutation by the same method described above.

### II. Peptides

Any method known in the art may be employed for the synthesis of peptides including but not limited to liquid phase, solid phase, or the use of recombinant organisms genetically engineered to express the selected polypeptide sequence. Peptides may be obtained from any number of commercial suppliers. Peptides once obtained may be used to prepare stock solutions where by they are dissolved in an appropriate solvent at concentrations to facilitate adding the peptide to a culture in an effective amount.

### A. Effective Amounts

With respect to effective amounts of autoinducing peptides the term “effective amount” is the amount of autoinducing peptide per liter that is required to manipulate or modify the various differentiated states of Clostridium in culture. That amount will vary depending on the particular autoinducing peptide, the particular strain of Clostridium, the culture conditions used, and the particular effect that is desired. It is expected that optimum effective amounts will be determined empirically. One of ordinary skill in the art will add an amount of peptide or peptides to the culture, and determine the degree and state of culture differentiation. It may be desirable to initiate cultures with an effective amount of autoinducing peptide and/or it may be desirable to monitor and maintain effective amounts of autoinducing peptides over a period of time. If desired, a sample of media may be removed from the culture and the concentration of autoinducing peptide analyzed through any method known in the art, for example by HPLC or immunochemical methods, and autoinducing peptides added accordingly. Examples of effective amounts of autoinducing peptide, expressed as amounts present in one liter, are expected to range from about 1 to about 100 picomoles, from about 100 to about 200 picomoles, from about 200 to about 300 picomoles, from about 300 to about 400 picomoles, from about 400 to about 500 picomoles, from about 500 to about 600 picomoles, from about 600 to about 700 picomoles, from about 700 to about 800 picomoles, from about 800 to about 900 picomoles or from about 900 to about 1000 picomoles, from about 1 to about 100 nanomoles, from about 100 to about 200 nanomoles, from about 200 to about 300 nanomoles, from about 300 to about 400 nanomoles, from about 400 to about 500 nanomoles, from about 500 to about 600 nanomoles, from about 600 to about 700 nanomoles, from about 700 to about 800 nanomoles, from about 800 to about 900 nanomoles or from about 900 to about 1000 nanomoles, from about 1 to about 100 micromoles, from about 100 to about 200 micromoles, from about 200 to about 300 micromoles, from about 300 to about 400 micromoles, from about 400 to about 500 micromoles, from about 500 to about 600 micromoles, from about 600 to about 700 micromoles, from about 700 to about 800 micromoles, from about 800 to about 900 micromoles or from about 900 to about 1000 micromoles. Preferably 100 picomoles to 1 micromole per liter. More preferably 1 nanomoles to 100 nanomoles per liter, and most preferably 10 nanomoles to 70 nanomoles per liter.

### B. Sequence Variation

It is well known that a certain amount of sequence variation may occur in polypeptides without affecting their function. It is expected that peptides closely resembling but not identical to the sequences disclosed herein may possess essentially the same function as their corresponding peptides or polypeptides and be used to practice the invention. It is expected that peptides or polypeptides with amino acid sequences which are 99 percent, 98 percent, 97 percent, 95 percent, 90 percent, 85 percent, 80 percent, 75 percent, 70 percent, 65 percent, 60 percent, 55 percent, or 50 percent identical to the autoinducing peptides or quorum sensing regulatory proteins disclosed herein may be used to practice the invention.

Sequence identity or “percent identity” is intended to mean the percentage of same residues between two sequences. In sequence comparisons, the two sequences being compared are aligned using the Clustal method (Higgins et al, (1992), Cabios, 8:189-191), of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NBRF, Washington, Vol. 5, suppl. 3, p. 345, 1978).

It is well-known in the biological arts that certain amino acid substitutions may be made in protein sequences without affecting the function of the protein. Generally, conservative amino acid substitutions or substitutions of similar amino acids are tolerated without affecting protein function. Similar amino acids can be those that are similar in size and/or charge properties, for example, aspartate and glutamate, and isoleucine and valine, are both pairs of similar amino acids. Similarity between amino acid pairs has been assessed in the art in a number of ways. For example, Dayhoff et al. (1978), in Atlas of protein Sequence and Structure, Volume 5, Supplement 3, Chapter 22, pp. 345-352, which is incorporated by reference herein, provides frequency tables for amino acid substitutions which can be employed as a measure of amino acid similarity. Dayhoff et al.'s frequency tables are based on comparisons of amino acid sequences for proteins having the same fraction from a variety of evolutionarily different sources.

It is also expected that less then the entire peptide or polypeptide sequence may possess essentially the same function as their corresponding autoinducing peptides or quorum sensing regulatory proteins disclosed herein. By way of example a polypeptide comprising any 5 consecutive or contiguous amino acids as set forth herein, may be used to practice the invention.

### D. Compositions

It is envisioned that certain compositions may facilitate the manipulation or modification of Clostridium cultures. Non-limiting examples include autoinducing peptides with amino acid sequences corresponding to natural occurring autoinducing peptides. Also included are autoinducing peptides with amino acid sequences derived in some way from natural occurring autoinducing peptides, including those with amino acid deletions or substitutions. Autoinducing peptides may be prepared alone or in combinations. Autoinducing peptides may be further combined with Clostridium organisms in any form, including growing organisms or spores. Autoinducing peptides may also be combined with any media capable of sustaining Clostridium cultures. Peptides with amino acid sequences corresponding to autoinducing peptides may be prepared in any formulation compatible with Clostridium culture. Such formulations may include autoinducing peptides in predetermined or effective amounts which manipulate or modify the various differentiated states of Clostridium in culture. Formulations may include sustained release formulations or formulations designed to release autoinducing peptides upon certain changes in the culture such as for example pH. Many such formulations are well known particularly to those skilled in the pharmaceutical or nutritional arts and may be easily adapted to Clostridium culture. Non-limiting examples are represented in U.S. Pat. Nos. 6,465,014 and 6,251,430 herein incorporated by reference in their entirety.

### III. Clostridium Cultures

### A. Clostridium

In general, the invention may be practiced on any strain of Clostridium of which an autoinducing peptide and/or quorum sensing regulatory proteins have been identified. For purposes of butanol fermentation any strain of Clostridium which forms primarily butanol may be employed. Preferred strains included Clostridium acetobutylicum ATCC 824, and Clostridium beijerinckii NCIMB 8052, which are available from the American Type Culture Collection, Rockville, Md. It is also expected that the invention may be practiced on any organisms which are within the same genetic lineage as C. acetobutylicum ATCC 824 or C. beijerinckii NCIMB 8052. Also included are organisms derived from C. acetobutylicum ATCC 824 or C. beijerinckii NCIMB 8052 by methods of genetic modification or other means. Non-limiting example of organisms within the same genetic lineage as Clostridium acetobutylicum include ATCC 824^{T} (=DSM 792^{T}=NRRL B527^{T}), ATCC 3625, DSM 1733 (=NCIMB 6441), NCIMB 6442, NCIMB 6443, ATCC 43084, ATCC 17792, DSM 1731 (=ATCC 4259=NCIMB 619=NRRL B530), DSM 1737, DSM 1732 (=NCIMB 2951), ATCC 39236, and ATCC 8529 (=DSM 1738). See Keis et al., (2001), International Journal of Systematic and Evolutionary Microbiology, 51: 2095-2103, incorporated herein in its entirety by reference. Non-limiting examples of organisms within the same genetic lineage as Clostridium beijerinckii include NCIMB 9362^{T}, NCIMB 11373, NCIMB 8052 (=DSM 1739=ATCC 10132=NRRL B594), NCIMB 8049, NCIMB 6444, NCIMB 6445, NCIMB 8653, NRRL 8591, NRRL B597, 214, 4J9, NCP 193, NCP 172(B), NCP 259, NCP 261, NCP 263, NCP 264, NCP 270, NCP 271, NCP 200(B), NCP 202(B), NCP 280, NCP 272(B), NCP 265(B), NCP 260, NCP 254(B), NCP 106, BAS/B/SW/136, BAS/B3/SW/336(B), BAS/B/136, ATCC 39058, NRRL B593, ATCC 17791, NRRL B592, NRRL B466, NCIMB 9503, NCIMB 9504, NCIMB 9579, NCIMB 9580, NCIMB 9581, NCIMB 12404, ATCC 17795, IAM 19015, ATCC 6014, ATCC 6015, ATCC 14823, ATCC 11914, and BA101. Id.

### B. Culture Methods

Typically the fermentation process is initiated by inoculating a seed culture or relatively small volume of sterile medium or distilled water under anaerobic conditions. The inoculum may be either Clostridium spores or active Clostridium organisms. The seed culture may allow the germination of spores and/or an increase in the initial number of organisms. The seed culture is then transferred to a larger volume of sterile media in a fermentor and fermented at a temperature from about 30° C. to about 40° C. Any type of Clostridium culture may be initiated using this method. Alternatively the fermentation vessel containing sterile medium may be inoculated directly.

Clostridium cultures may be subjected to any culture method or fermentation process known in the art, including but not limited to batch, fed batch or semi-continuous, continuous, or a combination of these processes. If batch culture or batch fermentation is employed, Clostridium cultures may be initiated as described above. The culture medium containing the inoculated organism may be fermented from about 30 hours to about 275 hours, preferably from about 45 hours to about 265 hours, at a temperature of from about 30° C. to about 40° C., preferably about 33° C. Preferably, sterilized nitrogen gas is sparged through the fermentor to aid mixing and to exclude oxygen.

If fed batch or semi-continuous culture or semi-continuous fermentation is employed, cultures may be initiated in the same manner as employed in batch fermentation, however after a period of time additional substrate is added to the fermentor. The culture medium containing the inoculated organism may then be fermented at a temperature from about 30° C. to about 40° C., preferably about 33° C. Sterile substrate may be added with or without monitoring the components of the culture. Growth rate may be controlled by the addition of substrate. Cultures may be initiated with lower amounts of initial substrate, and additional substrate feed to the reactor as the initial substrate is consumed. The use of fed batch or semi-continuous culture or fermentation may enable a higher yield of product from a given amount of substrate.

If continuous culture or continuous fermentation is employed, Clostridium cultures may be initiated as with other types of fermentation. The culture medium containing the inoculated organism may then be fermented at a temperature from about 30° C. to about 40° C., preferably about 33° C. Sterile medium flows into the fermentor and fermentation products and cells flow out. Fermentation products and cells may be easily harvested from the outflow. Cells and/or other components may be returned to the culture. The flow rate may very with the size of the inoculum, the concentration of carbohydrates and nutrients in the media, the rate of growth of the particular strain, and the rate of solvent production. It is expected that flow rates would be adjusted according to these culture parameters. Exemplary flow rates may be from 0.001 per hour to 0.50 per hour, preferably 0.005 per hour to 0.25 per hour, and most preferably 0.01 per hour to 0.1 per hour.

Other forms of continuous culture or continuous fermentation include two stage continuous cultures or two stage batch cultures as disclosed in U.S. Pat. Nos. 4,520,104 and 4,605,620 incorporated herein by reference. Generally these methods employ a first reactor to maintain an inoculum and a second reactor for fermentation. By this means, an inoculum produced in the first reactor is fed continuously into the second reactor where butanol production takes place. The continuous inoculum-producing reactor is run at a dilution rate which prevents the buildup of solvents in the medium thereby maintaining a culture of vital cells which is continuously transferred to the fermentation reactor. The fermentation reactor is also operated in a continuous mode but at a much lower dilution rate than the first reactor in which the inoculum is produced. The proper dilution rate in the fermentation reactor depends on the concentration of carbohydrate in the medium and the rate at which the medium is removed or recycled. For an efficient fermentation, the dilution and recycle rates are adjusted so that the carbohydrate is essentially all consumed.

### C. Culture Analysis and Culture Products

Regardless of the method of fermentation, samples may be removed routinely for analysis of any parameter including cell content, carbohydrate content, pH, organic acid, or solvent production. Cells may be analyzed using any method including but not limited to microscopy, optical density (O.D.), chemical, biochemical, or genetic analyses. Carbohydrate analysis may be conducted through any method known in the art including chemical, physical or enzyme based assays. The presence and concentration of autoinducing peptides may also be determined. The determination of peptides may be performed by any method known in the art including but not limited to the use of high pressure liquid chromatography (HPLC) and immunochemical including antibody and/or enzyme based methods including but not limited to Enzyme-linked immunosorbent assay (ELISA). Solvent and organic acid production may be detected using any chemical method known in the art including gas chromatography, HPLC, near infra red (NIR), or colorimetric methods, by way of example those based on ceric ammonium nitrate as described in Reid and Truelove, (1952), Analyst, 77, 325, incorporated herein in its entirety by reference.

In addition to butanol other products of fermentation may be harvested at any stage in the culture, including but not limited to: ethanol; propanol; isopropanol; 1,2 propanediol; 1,3 propanediol; amyl alcohol; isoamyl alcohol; hexanol; riboflavin; formic acid; acetic acid; butyric acid; lactic acid; formic, acetic butyric, lactic, caprylic, and capric esters of the alcohols; acetoin; acetone; biomass; CO₂; and hydrogen by any method known in the art. (for review see: Industrial Microbiology, S. C. Prescott and C. G. Dunn, McGraw-Hill Book Company, Inc., New York, 1940). In addition to products of fermentation other useful product may be harvested including bacteriocins, antibiotics, as well as various enzymes and amino acids. Cells may also be removed and returned to culture. The solvents, particularly, butanol, may be recovered using standard techniques known in the art. Non-limiting methods of harvesting butanol may include passing the media over an absorbent material such as activated carbon as described in U.S. Pat. Nos. 4,520,104, 327,849, and 2,474,170, incorporated herein in their entirety by reference, or passing the media over silicalite, as described in U.S. Pat. No. 5,755,967, incorporated herein in its entirety by reference.

### D. Culture Media

Regardless of the fermentation process employed, the Clostridium organism is inoculated and cultured on a medium containing assimilable carbohydrates and nutrients. Assimilable carbohydrates used in the practice of this invention may be any carbohydrate that will sustain or allow fermentation by the particular strain of Clostridium. These include solubilized starches and sugar syrups as well as glucose or sucrose in pure or crude forms. Assimilable carbohydrates also include glucose, maltodextrin, and corn steep liquor and hydrolyzed cellulosic substrates. Also included is glycerol. The culture medium should also contain nutrients and any other growth factors needed for growth and reproduction of the particular microorganism employed. By way of example but not of limitation commonly used commercially available media include P2, MP2, T6, TYA, TYG, TYGM, DMM, 2xYTG, RCA (Reinforced Clostridial Agar), RCM (Reinforced Clostridial Medium), RSM (Reinforced Soluble Medium), NYG (nutrient broth, yeast extract, glucose), CGM, CBM (Clostridial Basal Medium), PDM, PG (potato, glucose), and Cooked-meat medium. Optionally, the culture medium may contain one or more organic acids. Exemplary organic acids include acetic and butyric which may be added to the medium in exemplary amounts from about 20 mM to about 80 mM. The culture medium is preferably sterilized in the fermentor, agitated and sparged with nitrogen gas for about 12 hours to about 16 hours.

### Definitions

The term “differentiated state” or “differentiated states” as used herein, refers to a Clostridium organism, or a culture of Clostridium organisms, that are expressing a specialized function. Non-limiting examples of differentiated states or specialized functions include exponential growth, solventogenesis, acidogenesis, granulose synthesis, extended serial propagation, and sporogenesis.

The terms “manipulate or modify” as used herein in reference to differentiated states, refer to altering the usual behavior of Clostridium in any way, including but not limited to, enhancing or diminishing, or, changing or maintaining a differentiated state.

The term “exponential growth” as used herein, refers to a Clostridium organism or culture where the number of organisms is increasing exponentially. This may be determined by any number of methods known in the art including optical density (O.D.) of the culture media, or cell number as determined through counting or alike.

The term “solventogenesis” as used herein refers to a Clostridium organism, or culture where the organisms are producing solvents, including but not limited to any one or more of the following: ethanol, butanol, propanol, isopropanol, 1,2 propanediol, or acetone. Determination of solventogenesis may be performed by any number of methods known in the art including gas chromatography, high pressure liquid chromatography, or any method known to detect alcohols.

The term “acidogenesis” as used herein refers to a Clostridium organism, or culture where the organisms are producing organic acids, including but not limited to any one or more of the following: acetic acid, butyric acid, or lactic acid. Determination of acidogenesis may be performed by any method known in the art to detect organic acids, including gas chromatography, or high pressure liquid chromatography.

The terms “extending serial propagation,” or “extended serial propagation” as used herein, refers to the increased capacity for sequential inoculations, or sequential transfers from a Clostridium culture since the culture was derived from spores. This may also be expressed as an increased number of serial batch cultures serially inoculated from a Clostridium culture. The terms extending serial propagation, or extended serial propagation also refers to the increased length of time that a continuous culture of Clostridium may be maintained in a specific differentiated state without the addition of new inoculum. The terms extending serial propagation or extended serial propagation may also refer to an increased number of generations or population doublings by Clostridium organisms since being derived from spores.

The term “granulose synthesis” as used herein refers to a Clostridium organism, or culture, when the organisms synthesize carbohydrate storage granules. Determination of granulose synthesis may be performed by any known method including chemically, histological or microscopically. The skilled artisan will recognize clostridial storage cells microscopically, which are typically elongated and larger then cells not in involved granulose synthesis.

The term “sporogenesis” as used herein refers to a Clostridium organism, or culture, when the organisms form spores. Determination of sporogenesis may be performed by any known method including microscopically, chemically or genetically. The skilled artisan may recognize spores microscopically by a typical refractive appearance.

In addition to the various methods described above it is known that the differentiated states of Clostridium are the result of genetic and biochemical pathways. Therefore, the detection of any of the above differentiated states is not limited to the methods described herein but may be detected genetically, biochemically, immunochemically or by any method known in art.

The term “peptide” as used herein is meant to be synonymous with oligopeptide, polypeptide, or protein. The term peptide is meant to designate an amino acid polymer of 2 or more amino acids and is not meant to impose a limitation on the length of the amino acid polymer.

The term “autoinducing peptide” as used herein is meant to refer to any peptide that may manipulate or modify a differentiated state. The term autoinducing peptide is not limited to naturally occurring peptides, but may also refer to a peptide derived from naturally occurring peptides such as by amino acid substitution or deletion.

A “conservative amino acid substitution” is one in which an amino acid residue is replaced with another residue having a chemically similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, “percent Identity” of two amino acid sequences or of two nucleic acids is determined using the algorithm of Karlin and Altschul (Proc. Natl. Acad. Sci. USA, 87:2264-2268, 1990), modified as in Karlin and Altschul (Proc. Natl. Acad. Sci. USA, 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g. XBLAST and NBLAST) are used. See http://www.ncbi.nlm.nih.gov.

The term “dilution rate” as used herein, designates the value obtained by dividing the flow rate of the medium through the reactor in volume units per hour by the operating volume of the reactor measured in the same volume units. As stated, it has the implied dimensions of per hour.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Methods and Materials

Bacterial strains and media. Clostridium acetobutylicum ATCC 824 and C. beijerinckii NCIMB 8052 are available from several commercial microbial culture collections including the American Type Culture Collection (ATCC), Manassas, Va., USA. The strains were grown at 30° C. or 37° C. in YE broth, which contained, per liter: 5.0 g yeast extract, 2.5 g casamino acids, 1.0 g L-asparagine, 0.5 g cysteine.HCl, 56 mg K₂HPO₄, 56 mg KH₂PO₄, 82 mg anhydrous MgSO₄, 8 mg FeSO₄.H₂O, 6 mg MnSO₄.H₂O and 10 g glucose. Alternatively, strains were grown in YEPG broth, which was identical to YE expect that K₂HPO₄ and KH₂PO₄ were increased to 145 mg/L each and glucose was increased to 60 g/L. The pH of the media was adjusted to 7.2 using 45% KOH prior to sterilization by autoclaving. Media were solidified by addition of 1.5% Bacteriological Agar, Actimedia Manufacturers, Inc., Lansing, Mich. All cultures were grown in anaerobic conditions using the AnaeroPack System, Mitsubishi Gas Chemical Co., Inc., Japan, and GasPak EZ Gas Generating Sachets, Becton, Dickinson and Co., Sparks, Md. Spore stocks were kept at room temperature on agar-solidified media and were activated by suspending spores in 0.5 mL to 1.0 mL of medium followed by heating for 10 min at 80° C. before inoculation into growth medium.

Synthesis of peptides. Once peptides meeting the selection criteria were indentified putative autoinducing peptide sequences were chemically synthesized by a commercially available facility (Biomatik, Corp., Markham, Ontario, Canada) and were provided at >95% purity. Peptides were resuspended in an appropriate solvent, based on the peptide sequence, to give a 1 mM final concentration and were stored in small aliquots at −80° C. The peptides were diluted for use in experiments and were stored at 4° C. for one week before being discarded.

Growth and pH measurements. Growth of bacterial cultures was measured spectrophotometrically using optical density at 600 nm and pH of cell-free culture supernatants was measured using a hand-held Shindengen ISFET pH Meter KS501, Shendengen Electric Manufacturing Co., Ltd., Bannockburn, Ill.

Analysis of solvents. Total alcohols in cell-free culture supernatants were measured using a modification of a colorimetric method based on ceric ammonium nitrate (Reid and Truelove, 1952). The ceric ion reagent was prepared by adding 1.3 mL of concentrated nitric acid to 40 mL of distilled water, then 10.96 g of ceric ammonium nitrate was dissolved in the dilute nitric acid solution and the solution was brought to a final volume of 50 mL. For the assay, 100 μL of butanol standard or culture supernatant was mixed with 900 μL distilled water in a disposable plastic cuvette followed by addition of 400 μL of the ceric ion reagent. The sample was mixed by inverting the cuvette six times then exactly two minutes later the optical density at 500 nm wavelength was measured. The concentration of total alcohols was determined by comparison with a standard curve prepared by using butanol diluted in distilled water.

### Example 1

Identification of TPR repeat-containing proteins. Amino acid sequences of the quorum sensing protein family RNPP (Rap/NprR/PlcR/PrgX) were recovered from the online National Center for Biotechnology Information (NCBI) Protein database (Table 1)

TABLE 1
Proteins of the RNPP family of quorum
sensing regulatory proteins.

| SEQ ID NO | Protein | Organism | Accession |
|---|---|---|---|
| SEQ ID NO: 1 | PlcR | Bacillus thuringiensis | ZP_00739149 |
| SEQ ID NO: 2 | RapE | Bacillus thuringiensis | AAM51168 |
| SEQ ID NO: 3 | RapA | Bacillus thuringiensis | AAM51160 |
| SEQ ID NO: 4 | RapC | Bacillus subtilis | AAT75294 |
| SEQ ID NO: 5 | NprR | Bacillus thuringiensis | ABK83928 |
| SEQ ID NO: 6 | PrgX | Enterococcus faecalis | AAA65845 |
| SEQ ID NO: 7 | Treg | Enterococcus faecalis | NP_815038 |
| SEQ ID NO: 8 | DNAbd | Bacillus anthracis | NP_843644 |
| SEQ ID NO: 9 | TraA | Enterococcus faecalis | BAA11197 |
| SEQ ID NO: 10 | Tact | Listeria moncytogenes | YP_013453 |
| SEQ ID NO: 11 | Tre | Lactobacillus casei | YP_805489 |
| SEQ ID NO: 12 | RggD | Streptococcus gorondii | AAG32546 |
| SEQ ID NO: 13 | MutR | Streptococcus mutans | AAD56141 |

The RNPP family protein sequences were used separately as query sequences in Position-Specific Iterated (PSI)-Basic Local Alignment Search Tool (BLAST) alignments with the published genome sequences of C. beijerinckii NCIMB 8052 (NCBI Reference Sequence NC_{—}009617) (SEQ ID NO:14) and C. acetobutylicum ATCC 824 (NCBI Reference Sequence NC_{—}003030) (SEQ ID NO:15), and the C. acetobutylicum ATCC 824 plasmid pSOL1 sequence (NCBI Reference Sequence NC_{—}001988) (SEQ ID NO:16) using the online NCBI Position Specific Iterated—Basic Local Alignment Search Tool (PSI-BLAST) search engine. PSI-BLAST refers to a feature of BLAST 2.0 in which a profile, or position specific scoring matrix (PSSM), was constructed (automatically) from a multiple alignment of the highest scoring hits in an initial BLAST search. The PSSM was generated by calculating position-specific scores for each position in the alignment. Highly conserved positions receive high scores and weakly conserved positions receive scores near zero. The profile was used to perform subsequent searches. The BLAST search and the results of each “iteration” were used to refine the profile. This iterative searching strategy results in increased sensitivity (see Altschul, et al., (1997), Nucleic Acids Research; Vol. 25, No. 17, 3389-3402). A maximum of five Psi-Blast iterations were performed with each query sequence and alignments below the threshold value of 0.005 were considered to be matches.

Identification of putative secreted proteins associated with TPR repeat-containing proteins. Proteins identified in the genome sequences of C. beijerinckii NCIMB 8052 (NCBI Reference Sequence NC_{—}009617) (SEQ ID NO:14), C. acetobutylicum ATCC 824 (NCBI Reference Sequence NC_{—}003030) (SEQ ID NO:15) and C. acetobutylicum ATCC 824 plasmid pSOL1 (NCBI Reference Sequence NC_{—}001988) (SEQ ID NO:16), which aligned with members of the RNPP family, were examined using the NCBI Nucleotide Database Graphics format. Sequences of proteins in the same orientation which were immediately downstream from the identified protein sequences were recovered and analyzed for the presence of a typical Gram-positive secretion signal peptide. This process may be aided by the use of a Signal P 3.0 viewer which predicts the presence and location of secretion signal peptide cleavage sites in amino acid sequences. This method incorporates a prediction of cleavage sites and a signal peptide/non-signal peptide prediction based on a combination of several artificial neural networks and hidden models (see Bendtsen et al., (2004) J. of Mol. Biology, Vol. 340: 783-795). Proteins with secretion signal sequences were then examined for internal putative autoinducing peptides. Example 2

TPR repeat-containing proteins in C. acetobutylicum ATCC 824, C. beijerinckii NCIMB 8052 and C. acetobutylicum ATCC 824 plasmid pSOL1. A total of 46 individual protein sequences were identified in the C. acetobutylicum ATCC 824 genome and plasmid pSOL1 sequence by Psi-Blast alignments using RNPP family protein sequences as the queries (Table 2). PlcR and DNAbd aligned with nearly the same set of C. acetobutylicum proteins while RapC aligned with 9 members of that group and also with 20 additional proteins. NprR and Treg each aligned with a protein in the PlcR/DNAbd group, and Tact aligned with a protein that did not align with any of the other RNPP family members. The remaining 6 RNPP family proteins that were used as query sequences in Psi-Blast alignments did not align with any of the C. acetobutylicum proteins.

TABLE 2
RNPP family protein alignments with the C. acetobutylicum ATCC
824 genome (SEQ ID NO: 15) and plasmid pSOL1 (SEQ ID NO: 16).

| | NCIB | | Query Sequence | | | | | |
|---|---|---|---|---|---|---|---|---|
| SEQ ID NO | Reference | Locus Tag | PlcR | DNAbd | RapC | NprR | Treg | Tact |
| SEQ ID NO: 17 | NP_149204 | CA_P0040 | X | X | X | | | |
| SEQ ID NO: 18 | NP_347846 | CAC1214 | X | X | X | | | |
| SEQ ID NO: 19 | NP_346828 | CAC0186 | X | X | X | | | |
| SEQ ID NO: 20 | NP_149312 | CA_P0149 | X | X | X | | | |
| SEQ ID NO: 21 | NP_347679 | CAC1043 | X | X | X | | | |
| SEQ ID NO: 22 | NP_349104 | CAC2490 | X | X | X | | | |
| SEQ ID NO: 23 | NP_346965 | CAC0324 | X | X | X | | | |
| SEQ ID NO: 24 | NP_347593 | CAC0957 | X | X | X | | | |
| SEQ ID NO: 25 | NP_347594 | CAC0958 | X | X | | X | | |
| SEQ ID NO: 26 | NP_350275 | CAC3694 | X | X | X | | | |
| SEQ ID NO: 27 | NP_347477 | CAC0841 | X | X | | | | |
| SEQ ID NO: 28 | NP_350276 | CAC3695 | X | X | | | | |
| SEQ ID NO: 29 | NP_348569 | CAC1947 | X | X | | | X | |
| SEQ ID NO: 30 | NP_349841 | CAC3247 | X | X | | | | |
| SEQ ID NO: 31 | NP_350060 | CAC3472 | X | X | | | | |
| SEQ ID NO: 32 | NP_350228 | CAC3646 | X | X | | | | |
| SEQ ID NO: 33 | NP_348205 | CAC1578 | X | X | | | | |
| SEQ ID NO: 34 | NP_348467 | CAC1843 | X | X | | | | |
| SEQ ID NO: 35 | NP_349087 | CAC2473 | X | X | | | | |
| SEQ ID NO: 36 | NP_349109 | CAC2495 | X | X | | | | |
| SEQ ID NO: 37 | NP_349916 | CAC3324 | X | X | | | | |
| SEQ ID NO: 38 | NP_347105 | CAC0465 | X | | | | | |
| SEQ ID NO: 39 | NP_348186 | CAC1559 | X | X | | | | |
| SEQ ID NO: 40 | NP_348491 | CAC1867 | X | | | | | |
| SEQ ID NO: 41 | NP_348091 | CAC1463 | X | X | | | | |
| SEQ ID NO: 42 | NP_347698 | CAC1063 | | | X | | | |
| SEQ ID NO: 43 | NP_347702 | CAC1067 | | | X | | | |
| SEQ ID NO: 44 | NP_347699 | CAC1064 | | | X | | | |
| SEQ ID NO: 45 | NP_349230 | CAC2623 | | | X | | | |
| SEQ ID NO: 46 | NP_347052 | CAC0412 | | | X | | | |
| SEQ ID NO: 47 | NP_349426 | CAC2822 | | | X | | | |
| SEQ ID NO: 48 | NP_349599 | CAC2998 | | | X | | | |
| SEQ ID NO: 49 | NP_349900 | CAC3308 | | | X | | | |
| SEQ ID NO: 50 | NP_347561 | CAC0925 | | | X | | | |
| SEQ ID NO: 51 | NP_347056 | CAC0416 | | | X | | | |
| SEQ ID NO: 52 | NP_346692 | CAC0045 | | | X | | | |
| SEQ ID NO: 53 | NP_350039 | CAC3449 | | | X | | | |
| SEQ ID NO: 54 | NP_149324 | CA_P0161 | | | X | | | |
| SEQ ID NO: 55 | NP_348571 | CAC1949 | | X | X | | | |
| SEQ ID NO: 56 | NP_347055 | CAC0415 | | | X | | | |
| SEQ ID NO: 57 | NP_349405 | CAC2801 | | | X | | | |
| SEQ ID NO: 58 | NP_348952 | CAC2336 | | | X | | | |
| SEQ ID NO: 59 | NP_347044 | CAC0404 | | | X | | | |
| SEQ ID NO: 60 | NP_349017 | CAC2402 | | | X | | | |
| SEQ ID NO: 61 | NP_348298 | CAC1672 | | | X | | | |
| SEQ ID NO: 62 | NP_347555 | CAC0919 | | | | | | X |

### Example 3

A total of 28 individual protein sequences were identified in the C. beijerinckii NCIMB 8052 genome sequence by Psi-Blast alignments using RNPP family protein sequences as the queries (Table 3). PlcR, NprR and Treg aligned with nearly the same set of C. beijerinckii proteins, DNAbd aligned with a single protein in the PlcR/NprR/Treg group, and RapC aligned with a protein that did not align with any of the other RNPP family members. The remaining 7 RNPP family proteins that were used as query sequences in Psi-Blast alignments did not align with any of the C. beijerinckii proteins.

TABLE 3
RNPP family protein alignments with C. beijerinckii
NCIMB 8052 (SEQ ID NO: 14).

| | | | Query Sequence | | | | |
|---|---|---|---|---|---|---|---|
| SEQ ID NO | NCIB Reference | Locus Tag | PlcR | DNAbd | RapC | NprR | Treg |
| SEQ ID NO: 63 | YP_001307785 | Cbei_0642 | X | | | X | X |
| SEQ ID NO: 64 | YP_001310899 | Cbei_3827 | X | | | X | X |
| SEQ ID NO: 65 | YP_001310822 | Cbei_3749 | X | | | X | X |
| SEQ ID NO: 66 | YP_001308625 | Cbei_1492 | X | | | X | X |
| SEQ ID NO: 67 | YP_001309830 | Cbei_2723 | X | X | | X | X |
| SEQ ID NO: 68 | YP_001311025 | Cbei_3959 | X | | | X | X |
| SEQ ID NO: 69 | YP_001309285 | Cbei_2162 | X | | | X | X |
| SEQ ID NO: 70 | YP_001309337 | Cbei_2215 | X | | | X | X |
| SEQ ID NO: 71 | YP_001310692 | Cbei_3616 | X | | | X | X |
| SEQ ID NO: 72 | YP_001308745 | Cbei_1615 | X | | | X | X |
| SEQ ID NO: 73 | YP_001308026 | Cbei_0886 | X | | | X | X |
| SEQ ID NO: 74 | YP_001307786 | Cbei_0643 | X | | | X | X |
| SEQ ID NO: 75 | YP_001309382 | Cbei_2265 | X | | | X | X |
| SEQ ID NO: 76 | YP_001308393 | Cbei_1256 | X | | | X | X |
| SEQ ID NO: 77 | YP_001308072 | Cbei_0932 | X | | | X | X |
| SEQ ID NO: 78 | YP_001311244 | Cbei_4178 | X | | | X | X |
| SEQ ID NO: 79 | YP_001308109 | Cbei_0969 | X | | | X | X |
| SEQ ID NO: 80 | YP_001310559 | Cbei_3479 | X | | | X | X |
| SEQ ID NO: 81 | YP_001310563 | Cbei_3483 | X | | | X | X |
| SEQ ID NO: 82 | YP_001310537 | Cbei_3456 | X | | | X | X |
| SEQ ID NO: 83 | YP_001312058 | Cbei_4996 | X | | | X | X |
| SEQ ID NO: 84 | YP_001307844 | Cbei_0704 | X | | | X | X |
| SEQ ID NO: 85 | YP_001310808 | Cbei_3735 | X | | | | X |
| SEQ ID NO: 86 | YP_001312059 | Cbei_4997 | X | | | X | X |
| SEQ ID NO: 87 | YP_001310627 | Cbei_3549 | X | | | X | X |
| SEQ ID NO: 88 | YP_001307857 | Cbei_0717 | X | | | | |
| SEQ ID NO: 89 | YP_001308204 | Cbei_1064 | | | X | | |
| SEQ ID NO: 90 | YP_001307181 | Cbei_0035 | | | | X | X |

The total number of matches found in the genome sequences of C. acetobutylicum ATCC 824 and C. beijerinckii NCIMB 8052 with each query protein sequence is summarized in Table 4.

TABLE 4
Total number of matches found with each query protein sequence.

| | | | C. acetobutylicum |
|---|---|---|---|
| | Query | C. beijerinckii | SEQ ID NO: 15 and |
| SEQ ID NO | Sequence | SEQ ID NO: 14 | SEQ ID NO: 16 |
| SEQ ID NO: 1 | PlcR | 26 | 25 |
| SEQ ID NO: 2 | RapE | 0 | 0 |
| SEQ ID NO: 3 | RapA | 0 | 0 |
| SEQ ID NO: 4 | RapC | 1 | 29 |
| SEQ ID NO: 5 | NprR | 25 | 1 |
| SEQ ID NO: 6 | PrgX | 0 | 0 |
| SEQ ID NO: 7 | Treg | 26 | 1 |
| SEQ ID NO: 8 | DNAbd | 1 | 24 |
| SEQ ID NO: 9 | TraA | 0 | 0 |
| SEQ ID NO: 10 | Tact | 0 | 1 |
| SEQ ID NO: 11 | Tre | 0 | 0 |
| SEQ ID NO: 12 | Rggd | 0 | 0 |
| SEQ ID NO: 13 | MutR | 0 | 0 |

### Example 4

Putative secreted proteins associated with TPR repeat-containing proteins in C. acetobutylicum ATCC 824 and C. beijerinckii NCIMB 8052. The genomic regions and context of the sequence loci that were identified by Psi-Blast alignments with RNPP family protein sequences were examined with the aid of a graphic utility. Examples of such viewers include the Entrez Gene Sequence Viewer or MapViewer. In particular, genes immediately downstream from and transcribed in the same direction as the identified loci were identified. Thirty-three of the 45 loci identified in C. acetobutylicum and 19 of the 28 loci identified in C. beijerinckii had nearby downstream genes transcribed in the same direction (Tables 5 and 6).

TABLE 5
Genes immediately downstream from C. acetobutylicum ATCC
824 Psi-Blast alignments with RNPP family protein sequences.

| | | |
|---|---|---|
| SEQ ID NO | Locus Tag | Gene ID |

Aligned

| | | |
|---|---|---|
| SEQ ID NO: 17 | CA_P0040 | 1116045 |
| SEQ ID NO: 18 | CAC1214 | 1117397 |
| SEQ ID NO: 21 | CAC1043 | 1117226 |
| SEQ ID NO: 22 | CAC2490 | 1118673 |
| SEQ ID NO: 24 | CAC0957 | 1117140 |
| SEQ ID NO: 25 | CAC0958 | 1117141 |
| SEQ ID NO: 26 | CAC3694 | 1119876 |
| SEQ ID NO: 27 | CAC0841 | 1117024 |
| SEQ ID NO: 28 | CAC3695 | 1119877 |
| SEQ ID NO: 29 | CAC1947 | 1118130 |
| SEQ ID NO: 30 | CAC3247 | 1119429 |
| SEQ ID NO: 31 | CAC3472 | 1119654 |
| SEQ ID NO: 35 | CAC2473 | 1118656 |
| SEQ ID NO: 36 | CAC2495 | 1118678 |
| SEQ ID NO: 37 | CAC3324 | 1119506 |
| SEQ ID NO: 41 | CAC1463 | 1117646 |
| SEQ ID NO: 42 | CAC1063 | 1117246 |
| SEQ ID NO: 43 | CAC1067 | 1117250 |
| SEQ ID NO: 44 | CAC1064 | 1117247 |
| SEQ ID NO: 45 | CAC2623 | 1118806 |
| SEQ ID NO: 46 | CAC0412 | 1116595 |
| SEQ ID NO: 47 | CAC2822 | 1119005 |
| SEQ ID NO: 49 | CAC3308 | 1119490 |
| SEQ ID NO: 50 | CAC0925 | 1117108 |
| SEQ ID NO: 51 | CAC0416 | 1116599 |
| SEQ ID NO: 52 | CAC0045 | 1116228 |
| SEQ ID NO: 53 | CAC3449 | 1119631 |
| SEQ ID NO: 54 | CA_P0161 | 1116166 |
| SEQ ID NO: 56 | CAC0415 | 1116598 |
| SEQ ID NO: 57 | CAC2801 | 1118984 |
| SEQ ID NO: 58 | CAC2336 | 1118519 |
| SEQ ID NO: 59 | CAC0404 | 1116587 |
| SEQ ID NO: 61 | CAC1672 | 1117855 |

Downstream

| | | |
|---|---|---|
| SEQ ID NO: 91 | CA_P0039 | 1116044 |
| SEQ ID NO: 92 | CAC1215 | 1117398 |
| SEQ ID NO: 93 | CAC1044 | 1117227 |
| SEQ ID NO: 94 | CAC2488 | 1118671 |
| SEQ ID NO: 95 | CAC0958 | 1117141 |
| SEQ ID NO: 96 | CAC0959 | 1117142 |
| SEQ ID NO: 97 | CAC3693 | 1119875 |
| SEQ ID NO: 98 | CAC0840 | 1117023 |
| SEQ ID NO: 99 | CAC3694 | 1119876 |
| SEQ ID NO: 100 | CAC1948 | 1118131 |
| SEQ ID NO: 101 | CAC3246 | 1119428 |
| SEQ ID NO: 102 | CAC3470 | 1119652 |
| SEQ ID NO: 103 | CAC2474 | 1118657 |
| SEQ ID NO: 104 | CAC2494 | 1118677 |
| SEQ ID NO: 105 | CAC3323 | 1119505 |
| SEQ ID NO: 106 | CAC1464 | 1117647 |
| SEQ ID NO: 107 | CAC1064 | 1117247 |
| SEQ ID NO: 108 | CAC1068 | 1117251 |
| SEQ ID NO: 109 | CAC1065 | 1117248 |
| SEQ ID NO: 110 | CAC2622 | 1118805 |
| SEQ ID NO: 111 | CAC0413 | 1116596 |
| SEQ ID NO: 112 | CAC2821 | 1119004 |
| SEQ ID NO: 113 | CAC3307 | 1119489 |
| SEQ ID NO: 114 | CAC0926 | 1117109 |
| SEQ ID NO: 115 | CAC0417 | 1116600 |
| SEQ ID NO: 116 | CAC0046 | 1116229 |
| SEQ ID NO: 117 | CAC3450 | 1119632 |
| SEQ ID NO: 118 | CA_P0162 | 1116167 |
| SEQ ID NO: 119 | CAC0416 | 1116599 |
| SEQ ID NO: 120 | CAC2800 | 1118983 |
| SEQ ID NO: 121 | CAC2335 | 1118518 |
| SEQ ID NO: 122 | CAC0405 | 1116588 |
| SEQ ID NO: 123 | CAC1673 | 1117856 |

TABLE 6
Genes immediately downstream from C. beijerinckii NCIMB 8052
Psi-Blast alignments with RNPP family protein sequences.

| | | |
|---|---|---|
| SEQ ID NO | Locus Tag | Gene ID |

Aligned

| | | |
|---|---|---|
| SEQ ID NO: 63 | Cbei_0642 | 5291873 |
| SEQ ID NO: 64 | Cbei_3827 | 5294989 |
| SEQ ID NO: 65 | Cbei_3749 | 5294912 |
| SEQ ID NO: 66 | Cbei_1492 | 5292713 |
| SEQ ID NO: 67 | Cbei_2723 | 5293919 |
| SEQ ID NO: 68 | Cbei_3959 | 5295115 |
| SEQ ID NO: 71 | Cbei_3616 | 5294782 |
| SEQ ID NO: 73 | Cbei_0886 | 5292114 |
| SEQ ID NO: 74 | Cbei_0643 | 5291874 |
| SEQ ID NO: 76 | Cbei_1256 | 5292481 |
| SEQ ID NO: 80 | Cbei_3479 | 5294649 |
| SEQ ID NO: 81 | Cbei_3483 | 5294653 |
| SEQ ID NO: 82 | Cbei_3456 | 5294627 |
| SEQ ID NO: 85 | Cbei_3735 | 5294898 |
| SEQ ID NO: 86 | Cbei_4997 | 5296149 |
| SEQ ID NO: 87 | Cbei_3549 | 5294717 |
| SEQ ID NO: 88 | Cbei_0717 | 5291945 |
| SEQ ID NO: 89 | Cbei_1064 | 5292292 |
| SEQ ID NO: 90 | Cbei_0035 | 5291269 |

Downstream

| | | |
|---|---|---|
| SEQ ID NO: 124 | Cbei_0643 | 5291874 |
| SEQ ID NO: 125 | Cbei_3826 | 5294988 |
| SEQ ID NO: 126 | Cbei_3748 | 5294911 |
| SEQ ID NO: 127 | Cbei_1491 | 5292712 |
| SEQ ID NO: 128 | Cbei_2722 | 5293918 |
| SEQ ID NO: 129 | Cbei_3960 | 5295116 |
| SEQ ID NO: 130 | Cbei_3615 | 5294781 |
| SEQ ID NO: 131 | Cbei_0885 | 5292113 |
| SEQ ID NO: 132 | Cbei_0644 | 5291875 |
| SEQ ID NO: 133 | Cbei_1257 | 5292482 |
| SEQ ID NO: 134 | Cbei_3478 | 5294648 |
| SEQ ID NO: 135 | Cbei_3482 | 5294652 |
| SEQ ID NO: 136 | Cbei_3455 | 5294626 |
| SEQ ID NO: 137 | Cbei_3734 | 5294897 |
| SEQ ID NO: 138 | Cbei_4998 | 5296150 |
| SEQ ID NO: 139 | Cbei_3550 | 5294718 |
| SEQ ID NO: 140 | Cbei_0718 | 5291946 |
| SEQ ID NO: 141 | Cbei_1065 | 5292293 |
| SEQ ID NO: 142 | Cbei_0036 | 5291270 |

Each of the protein sequences for the downstream proteins listed in Tables 5 and 6, above, was analyzed for the presence of a typical Gram-positive protein secretion signal peptide using the Signal P 3.0 server (see Bendtsen et al., (2004) J. of Mol. Biology, 340: 783-795). Four of the 33 downstream proteins in C. acetobutylicum ATCC 824 had putative secretion signals, while only 1 of the downstream proteins in C. beijerinckii NCIMB 8052 contained a secretion signal (Table 7).

TABLE 7
Proteins immediately downstream from RNPP-aligned proteins
in C. acetobutylicum ATCC 824 and C. beijerinckii NCIMB
8052 that contain putative secretion signals.

| | | Probability | | Length | |
|---|---|---|---|---|---|
| | | Signal | Cleavage | Signal | Released |
| SEQ ID NO | Locus Tag | Peptide | Site | Sequence | Protein |
| SEQ ID NO: 97 | CAC3693 | 0.995 | 0.997 | 34 aa | 7 aa |
| SEQ ID NO: 110 | CAC2622 | 0.997 | 0.577 | 32 aa | 275 aa |
| SEQ ID NO: 112 | CAC2821 | 0.727 | 0.385 | 29 aa | 649 aa |
| SEQ ID NO: 121 | CAC2335 | 0.639 | 0.638 | 23 aa | 280 aa |
| SEQ ID NO: 141 | Cbei_1065 | 0.999 | 0.999 | 25 aa | 152 aa |

### Example 5

Identification of autoinducing peptides in putative secreted proteins. C. acetobutylicum ATCC 824 locus CAC3693 (SEQ ID NO: 97) has been described as a hypothetical protein in the genome sequence of that organism. The 5′ end of the proposed coding sequence for CAC3693 overlaps 8 nucleotides of the 3′ end of the upstream TPR repeat-containing protein CAC3694 (SEQ ID NO: 26), which was identified by alignment of PlcR, RapC and DNAbd with the C. acetobutylicum genome using Psi-Blast. CAC3693 is likely exported from the cell by means of the putative secretion signal, and cleavage of the signal sequence would then release a heptapeptide with the amino acid sequence SYPGWSW (SEQ ID NO:143). The genetic organization of the TPR repeat-containing CAC3694 and the overlapping downstream, secreted CAC3693 is reminiscent of that of the Rap protein and associated Phr peptide genes in Bacillus subtilis, which encode phosphatases and phosphatase inhibitors, respectively (Perego, Peptides 22:1541-1547, 2001). While the B. subtilis Phr peptides can be aligned on a RxxT amino acid sequence motif or on an internal lysine residue, the sequence identified in C. acetobutylicum is quite different and contains 2 tryptophan residues.

C. acetobutylicum ATCC 824 locus CAC2622 (SEQ ID NO: 110) has been described as a ComE-like protein. The 5′ end of the coding sequence for the protein is located about 250 nucleotides downstream from the end of CAC2623 (SEQ ID NO: 45), which has been described as a quorum sensing regulatory protein and was identified in this study by alignment with RapC. As a ComE-like protein, CAC2622 might be involved with DNA binding or uptake at the cell surface. CAC2622 is likely exported from the cell and the secretion signal peptide is cleaved as a 32, 30, or 23 amino acid leader. A cysteine residue located at position 24 of the protein, immediately distal to a possible leader peptide cleavage site, is somewhat reminiscent of the structure of Enterococcal autoinducing precursors (Clewell, Mol Microbiol 35:246-247, 2000). CAC2622 is likely exported from the cell by means of the putative secretion signal, and further processing of the signal sequence would then release a heptapeptide with the amino acid sequence ILILISG (SEQ ID NO:144).

A BLAST search of the C. acetobutylicum ATCC 824 plasmid pSOL1 sequence (SEQ ID NO:16) using the heptapeptide ILILISG (SEQ ID NO:144) as the query found a similar protein sequence located in the putative protein CA_P0131 (SEQ ID NO:146), which is described as a relative of the multidrug resistance protein family. Also, Signal P 3.0 identified an N-terminal putative protein secretion signal making it likely that CA_P0131 is exported from the cell. Further processing of the protein would then release a peptide with an amino acid sequence similar to SEQ ID NO:144,

C. beijerinckii NCIMB 8052 locus Cbei_{—}1065 (SEQ ID NO: 141) has been described as a hypothetical protein in the genome sequence of that organism. The 5′ end of the coding sequence for the protein is located about 640 nucleotides downstream from the end of Cbei_{—}1064 (SEQ ID NO: 89), which is described as a TPR repeat-containing protein and was identified by alignment with RapC. The N-terminal sequence of Cbei_{—}1065 contains a typical Gram-positive signal sequence that would result in export and release of a 152 amino acid protein. The remaining 25 amino acid secretion signal contains a Phr peptide RxxT motif, and further processing of the leader peptide could release the pentapeptide IRLIT (SEQ ID NO:145).

A BLAST search of the C. beijerinckii NCIMB genome sequence (SEQ ID NO:14) using the pentapeptide IRLIT (SEQ ID NO:145) as the query found an identical protein sequence located in the putative protein Cbei_{—}2139 (SEQ ID NO:147). Cbei_{—}2139 has been described as a transport system permease protein. Signal P 3.0 identified an N-terminal putative protein secretion signal making it likely that Cbei2139 is exported from the cell by means of the putative secretion signal. Further processing of the protein would then release a peptide that contains an amino acid sequence similar to SEQ ID NO:145. Peptides and putative proteins from C. acetobutylicum ATCC 824 and C. beijerinckii NCIMB 8052 that might function as or contain autoinducing peptides are summarized in Table 8.

C. beijerinckii NCIMB locus Cbei_{—}1066 (SEQ ID NO:148) has also been described as a hypothetical protein in the genome sequence of that organism. The 5′ end of the coding sequence for the protein is located about 905 nucleotides downstream from the end of Cbei_{—}1065 (SEQ ID NO:145). The N-terminal sequence of Cbei_{—}1066 appears to contain a typical Gram-positive signal sequence that would result in export and release of a 176 amino acid protein and a 27 amino acid secretion signal. Further processing of either the released protein or secretion signal may result in release of a peptide that functions as a quorum sensor.

TABLE 8
Autoinducing Peptides from C. acetobutylicum ATCC 824 and
C. beijerinckii NCIMB 8052.

| | | Autoinducing Peptide | |
|---|---|---|---|
| | | SEQ ID | |
| Organism | Locus | NO | Sequence |
| C. | CAC3693 | SEQ ID | SYPGWSW |
| acetobutylicum | | NO: 143 | |
| C. | CAC2622 | SEQ ID | ILILISG |
| acetobutylicum | | NO: 144 | |
| C. beijerinckii | Cbei_1065 | SEQ ID | IRLIT |
| | | NO: 145 | |
| C. | CA_P0131 | SEQ ID | MTQMNSRKKSIIASLMVAMFLGAIEGTVVTTA |
| acetobutylicum | | NO: 146 | MPTIVRDLNGFDKISLVFSVYLLTSAISTPIYG |
| | | | KIADLYGRKRALSTGIIIFLLGSALCGISSNMY |
| | | | ELILFRALQGIGAGSIFTVSYTIVGDVFSLEER |
| | | | GKVQGWISSVWGIASLLGPFIGGFFIDYMSW |
| | | | NWIFYINLPFGIFSLVLLEKNLKEKVEKKKTPM |
| | | | DYLGIVTLTLTIVIFLLTILGINENTKISSAKIILP |
| | | | MLVTVLLLFVFYFIEKRAKEPLIPFDIFSKQSNI |
| | | | VNIISFLVSGILIGTDVYLPIYIQNVLGYSATISG |
| | | | LSLASMSISWILSSFVLSKAIQKYGERPVVFIS |
| | | | TLITLVSTVLFYTLTGNSPLILVIIYGFIIGFGYG |
| | | | GTLTTLTIVIQEAVSKDKRGAATGANSLLRTM |
| | | | GQTIGVAIFGVIFNLNIAKYLYKLGIRGINVNSL |
| | | | YGSGNVHTGIPLDKVKASLNFGVHTLFFILILI |
| | | | SVICTIMSVMLSNSLNKKKNMR |
| C. beijerinckii | Cbei_2139 | SEQ ID | MKRNNKNAITFTVCSIFILIVGLILGVSLGATQI |
| | | NO: 147 | GISEIWHSIFNYSERLELVLIRDVRIPRVLCVL |
| | | | FTGGILGVTGAMIQGVTRNPIAEPSLLGVSQ |
| | | | GATLVIAIFYAMGISINTTNVMIAALIGSIFSGII |
| | | | VIGFISKKANNSSITKILLAGTAMSTFFISLTTIV |
| | | | GLLSNQSQLLAFWVAGGFRNATWLDFKLVS |
| | | | VIATIGLIIALLLSKKINILSLGDDVAISLGQNPE |
| | | | KIRLITLLVMIPMCAGAVAVGKNIGFVGLIVPQI |
| | | | VRKILGEDYRINIPCSFLLGAVLLTYADIAARM |
| | | | FLNPYETPIGIFTALIGVPFFIAVARKEKG |
| C. beijerinckii | Cbei_1066 | SEQ ID | MTRKLIIATVLMLSTVMVSCSTKPSDSPKPSD |
| | | NO: 148 | NNTTTVEQNKDDNGSSNADSKKANETTSDT |
| | | | KKVNKVKLSIYSIDDNSLEPNESGTIEVNENS |
| | | | ALQDKLKELAKAVSEKKFDNLPIEVKSIDTVN |
| | | | GKKVATINLTDSNNKKWVPKFQGSTGGSVT |
| | | | ANTLIENFLQSNNKSKGEWIDGVKFLYNNETI |
| | | | EYEHASDLSTVKYAN |

### Example 6

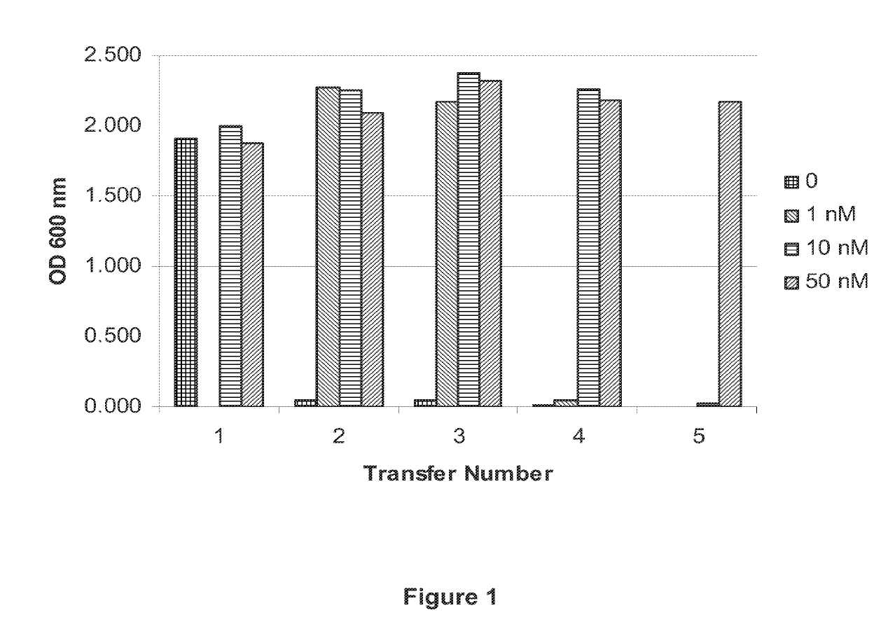

Effect of peptide SEQ ID NO:143 addition on sequential batch cultures of C. acetobutylicum ATCC 824 grown at 30° C. Spores of C. acetobutylicum ATCC 824 were germinated and grown overnight at 30° C. under anaerobic conditions in YEPG medium. After about 24 h of growth, 75 μL of the culture was transferred (transfer 1) to each of four flasks that contained 10 mL of YEPG and either had no treatment or were treated with peptide SEQ ID NO:143 (see Table 8 and FIG. 1) at 1 nM, 10 nM or 50 nM. Thereafter, 75 μL of each culture was transferred, at the same time, every 24-48 h to 10 mL of fresh YEPG that contained the same peptide treatment or no treatment. Each culture was stopped after 96 hours of incubation and optical density, pH and ceric ion reactive chemicals were measured. Sequential batch culturing was continued through 5 transfers at which point the untreated culture and those treated with 1 nM and 10 nM of peptide SEQ ID NO:143 had stopped growing (Table 9). The untreated culture did not grow after the second transfer, but growth was prolonged past the second transfer for all cultures treated with peptide SEQ ID NO:143. The peptide treatments showed a dose response for extending growth during sequential batch cultures in that adding peptide SEQ ID NO:143 to 1 nM allowed growth through the third transfer, 10 nM allowed growth through the fourth transfer and 50 nM extended growth through the fifth transfer. In addition, treatment with 1 nM of peptide SEQ ID NO:143 appeared to stop growth at the first transfer, but growth was restored in the second and third transfers.

TABLE 9
Optical density at 600 nm of C. acetobutylicum ATCC
824 96 h culture broths following sequential transfers
in the absence and presence of peptide SEQ ID NO: 143.

Peptide SEQ ID NO: 143 Concentration

| Transfer | 0 | 1 nM | 10 nM | 50 nM |
|---|---|---|---|---|
| 1 | 1.908 | 0.005 | 2.001 | 1.879 |
| 2 | 0.043 | 2.274 | 2.245 | 2.089 |
| 3 | 0.042 | 2.165 | 2.379 | 2.313 |
| 4 | 0.007 | 0.044 | 2.266 | 2.187 |
| 5 | 0.004 | 0.004 | 0.028 | 2.173 |

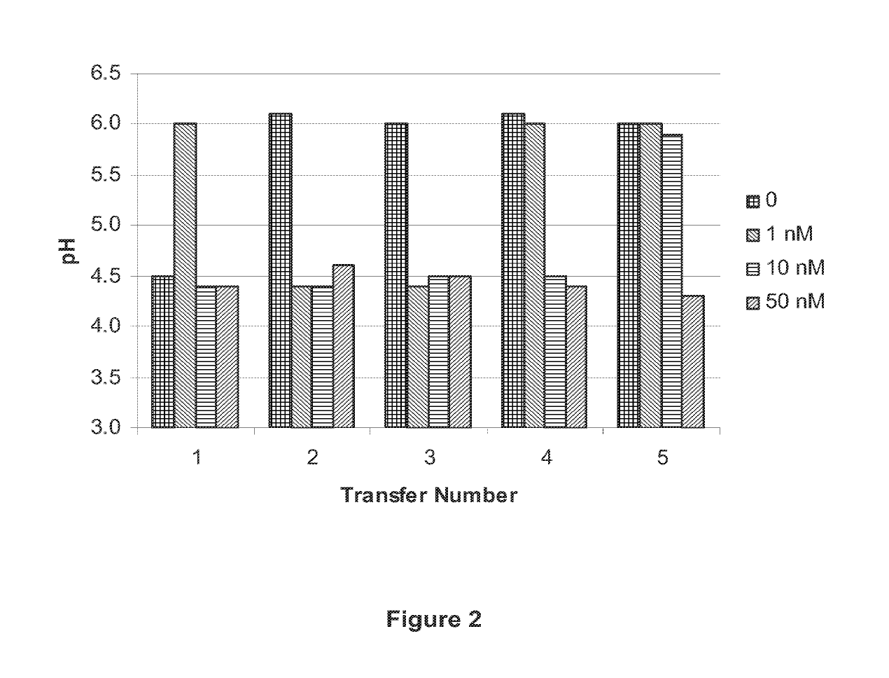

Final pH of the sequential cultures mirrored the growth results (Table 10 and FIG. 2). Cultures that grew had final pH values, after 96 h, of 4.6 or less while cultures that did not grow had final pH readings of 5.9 and higher For the untreated culture, final pH rose to 6.1 at the second transfer while the final pH of cultures treated with 1 nM and 10 nM of peptide SEQ ID NO:143 rose to 6.0 and 5.9 after the fourth and fifth transfers, respectively. The pH of the culture treated with 50 nM of peptide SEQ ID NO:143 remained low at the fifth transfer. Also reflecting the optical density data, the final pH of the culture treated with 1 nM of peptide SEQ ID NO:143 was 6.0 at the first transfer but then dropped to 4.4 at the second and third transfers.

TABLE 10
Final pH of C. acetobutylicum ATCC 824 96 h culture
broths following sequential transfers in the absence
and presence of peptide SEQ ID NO: 143.

Peptide SEQ ID NO: 143 Concentration

| Transfer | 0 | 1 nM | 10 nM | 50 nM |
|---|---|---|---|---|
| 1 | 4.5 | 6.0 | 4.4 | 4.4 |
| 2 | 6.1 | 4.4 | 4.4 | 4.6 |
| 3 | 6.0 | 4.4 | 4.5 | 4.5 |
| 4 | 6.1 | 6.0 | 4.5 | 4.4 |
| 5 | 6.0 | 6.0 | 6.0 | 4.3 |

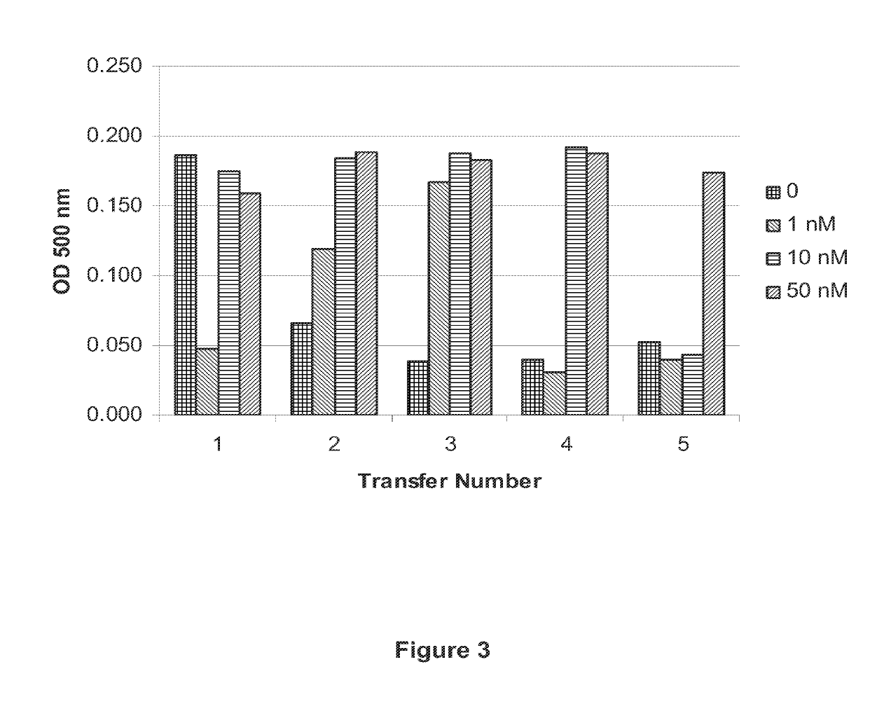

The presence of ceric ion reactive chemicals, which reflects total alcohols concentration in the fermentation broths, was also affected by the addition of peptide SEQ ID NO:143 in sequential batch cultures (Table 11 and FIG. 3). While ceric ion reactive compounds decreased in the untreated culture and the cultures treated with 1 nM and 10 nM peptide SEQ ID NO:143 they did not decrease through five sequential transfers of the culture treated with 50 nM. Similar to the dose response seen in the growth data (see Table 9 and FIG. 1), ceric ion reactive compounds decreased dramatically at the second transfer of the untreated culture and at the fourth and fifth transfers of the cultures treated with 1 nM and 10 nM of peptide SEQ ID NO:143, respectively. Also reflecting the optical density data, the presence of ceric ion reactive compounds was low in the culture treated with 1 nM of peptide SEQ ID NO:143 at the first transfer but then increased at the second and third transfers.

TABLE 11
Optical density of ceric ion reactive compounds measured
at 500 nm in C. acetobutylicum ATCC 824 96 h culture
broths following sequential transfers in the absence
and presence of peptide SEQ ID NO: 143.

Peptide SEQ ID NO: 143 Concentration

| Transfer | 0 | 1 nM | 10 nM | 50 nM |
|---|---|---|---|---|
| 1 | 0.186 | 0.048 | 0.175 | 0.159 |
| 2 | 0.066 | 0.119 | 0.184 | 0.189 |
| 3 | 0.039 | 0.167 | 0.187 | 0.183 |
| 4 | 0.040 | 0.031 | 0.192 | 0.187 |
| 5 | 0.052 | 0.040 | 0.043 | 0.174 |

In summary, addition of peptide SEQ ID NO:143 to broth cultures of C. acetobutylicum ATCC 824 allowed the cultures to be sequentially transferred at least four more times than a culture that did not receive added peptide. The production of alcohols, shown by ceric ion reactive compounds, continued through the sequential transfers and did not decrease until transfer was unsuccessful. In addition, the number of sequential transfers showed a dose response in relation to the concentration of added peptide with the highest concentration surviving the most transfers. Addition of peptide SEQ ID NO:143 was able to prevent culture degeneration in terms of the number of sequential transfers and production of total alcohols.

Under these experimental conditions, and knowledge of the growth of C. acetobutylicum in culture, it was determined that each sequential transfer was equivalent to about seven bacterial generations (Kashket, Applied and Environmental Microbiology 59:4198-4202, 1993). In other words, the first transfer took place after about seven bacterial generations and by the fifth transfer about 35 bacterial generations have been completed. The number of population doublings or bacterial generations observed in batch culture is expected to be comparable in continuous culture. From these results, an estimate of extended serial propagation in continuous culture may be made from the sequential batch transfers in batch culture, and the expected number of population doublings or bacterial generations per transfer. An estimate of extended serial propagation in continuous culture may be expressed as extended time in continuous culture by taking the dilution rate into account. In continuous culture, the time for one generation is equal to the inverse of the dilution rate. Accordingly, it may be expected from the above data, that the addition of peptide SEQ ID NO: 143 to C. acetobutylicum in continuous culture, maintained at a dilution rate of 0.05/hour, would extend the time in culture about five-fold from about 140 hours to about 700 hours.

### Example 7

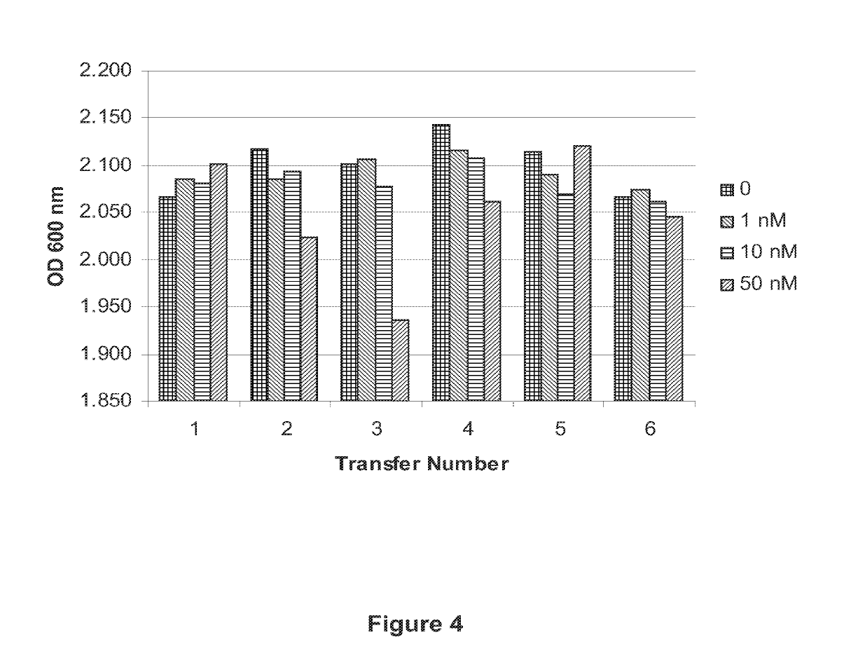

Effect of peptide SEQ ID NO:145 addition on sequential batch cultures of C. beijerinckii NCIMB 8052 grown at 30° C. Spores of C. beijerinckii NCIMB 8052 were germinated and grown overnight at 30° C. under anaerobic conditions in YEPG medium. After about 24 h of growth, 754 of the culture was transferred (transfer 1) to each of four flasks that contained 10 mL of YEPG and either had no treatment or were treated with peptide SEQ ID NO:145 (see Table 8) at 1 nM, 10 nM or 50 nM. Thereafter, 75 μL of each culture was transferred, at the same time, every 24-48 h to 10 mL of fresh YEPG that contained the same peptide treatment or no treatment. Each culture was stopped after 96 hours of incubation and optical density, pH and ceric ion reactive chemicals were measured. Sequential batch culturing was continued through 6 transfers at which point all cultures appeared to be growing to the same extent (Table 12 and FIG. 4). However, addition of peptide SEQ ID NO:145 appeared to slow the growth of the treated cultures during 96 h of incubation in a dose dependent manner (data not shown). Also, addition of 50 nM peptide SEQ ID NO:145 slightly decreased the final optical density of transfers two and three, compared to the other three cultures, and the optical density increased to values similar to the other cultures by transfers five and six.

TABLE 12
Optical density at 600 nm of C. beijerinckii NCIMB 8052
96 h culture broths following sequential transfers in the
absence and presence of peptide SEQ ID NO: 145.

Peptide SEQ ID NO: 145 Concentration

| Transfer | 0 | 1 nM | 10 nM | 50 nM |
|---|---|---|---|---|
| 1 | 2.066 | 2.086 | 2.080 | 2.102 |
| 2 | 2.117 | 2.086 | 2.093 | 2.023 |
| 3 | 2.101 | 2.106 | 2.078 | 1.936 |
| 4 | 2.142 | 2.115 | 2.108 | 2.061 |
| 5 | 2.114 | 2.090 | 2.069 | 2.120 |
| 6 | 2.066 | 2.075 | 2.062 | 2.046 |

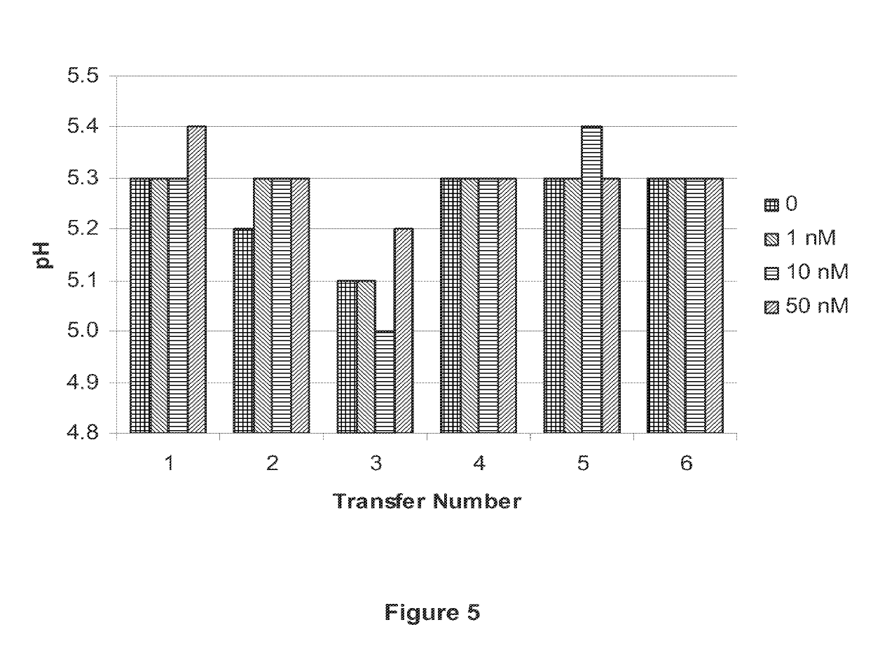

Final pH values of the fermentation broths did not mirror the growth data as measured by optical density (Table 13 and FIG. 5). While the final pH of all cultures decreased through the third transfer, the pH of the culture treated with 10 nM peptide SEQ ID NO 145 was the lowest at the third transfer while the pH of the culture treated with 50 nM was the highest. After the third transfer, final pH values of all cultures rose and stayed at about pH 5.3.

TABLE 13
Final pH of C. beijerinckii NCIMB 8052 96 h culture
broths following sequential transfers in the absence
and presence of peptide SEQ ID NO: 145.

Peptide SEQ ID NO: 145 Concentration

| Transfer | 0 | 1 nM | 10 nM | 50 nM |
|---|---|---|---|---|
| 1 | 5.3 | 5.3 | 5.3 | 5.4 |
| 2 | 5.2 | 5.3 | 5.3 | 5.3 |
| 3 | 5.1 | 5.1 | 5.0 | 5.2 |
| 4 | 5.3 | 5.3 | 5.3 | 5.3 |
| 5 | 5.3 | 5.3 | 5.4 | 5.3 |
| 6 | 5.3 | 5.3 | 5.3 | 5.3 |

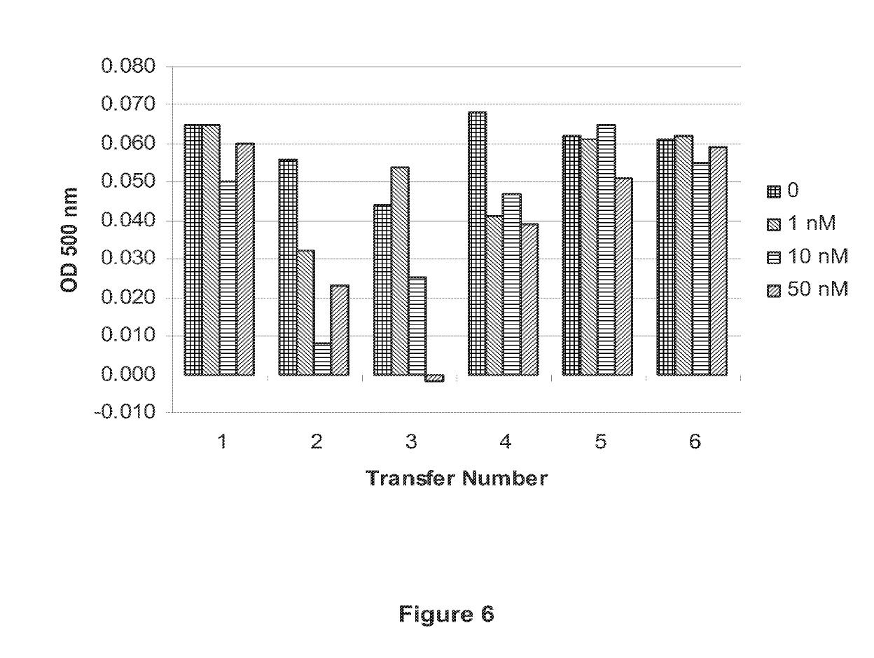

The presence of ceric ion reactive chemicals, which reflects total alcohols concentration in the fermentation broths, was also affected by the addition of peptide SEQ ID NO:145 in sequential batch cultures (Table 14 and FIG. 6). Cultures treated with peptide SEQ ID NO:145 all showed pronounced decreases in ceric ion reactive compounds which rebounded to the level observed in the untreated cultures by the fifth and sixth transfers. While the cultures treated with 1 nM and 10 nM of peptide SEQ ID NO:145 had their lowest values at transfer 2, and then increased with subsequent transfers, the culture treated with 50 nM continued decreasing after transfer 2 and had no ceric ion reactive compounds at transfer 3. The impact of peptide SEQ ID NO:145 treatment also had a dose response effect on ceric ion reactive compounds such that the 50 nM treatment reached the lowest value overall, the 10 nM treatment was next lowest and the 1 nM treatment was next but still lower than the untreated cultures.

TABLE 14
Optical density of ceric ion reactive compounds measured
at 500 nm in C. beijerinckii NCIMB 8052 96 h culture
broths following sequential transfers in the absence
and presence of peptide SEQ ID NO: 145.

Peptide SEQ ID NO: 145 Concentration

| Transfer | 0 | 1 nM | 10 nM | 50 nM |
|---|---|---|---|---|
| 1 | 0.065 | 0.065 | 0.050 | 0.060 |
| 2 | 0.056 | 0.032 | 0.008 | 0.023 |
| 3 | 0.044 | 0.054 | 0.025 | −0.002 |
| 4 | 0.068 | 0.041 | 0.047 | 0.039 |
| 5 | 0.062 | 0.061 | 0.065 | 0.051 |
| 6 | 0.061 | 0.062 | 0.055 | 0.059 |

Addition of peptide SEQ ID NO:145 to broth cultures of C. beijerinckii NCIMB 8052 did not affect the number of times that cultures could be transferred, through six culture transfers, in comparison with an untreated culture. Peptide treatment slightly decreased end point growth measurements through the fourth transfer and that was most evident in cultures that had the highest peptide concentration. In addition, the peptide treatments slowed the growth of cultures in a dose dependent manner through the 96 h incubation period (data not shown). Finally, the presence of ceric ion reactive compounds was decreased in peptide-treated cultures through the fourth transfer, and the greatest decrease was seen in cultures with the highest peptide concentration. Ceric ion reactive compounds in peptide-treated cultures returned to about the same level as in untreated cultures by the sixth transfer. In this case, peptide treatment seemed to transiently increase culture degeneration in terms of production of total alcohols: Therefore, the gene sequence that encodes peptide SEQ ID NO: 145 is a potential candidate for genetic modification to reduce or eliminate formation of the peptide, which should reduce or eliminate the antagonistic effect on growth and butanol formation.

### Example 8

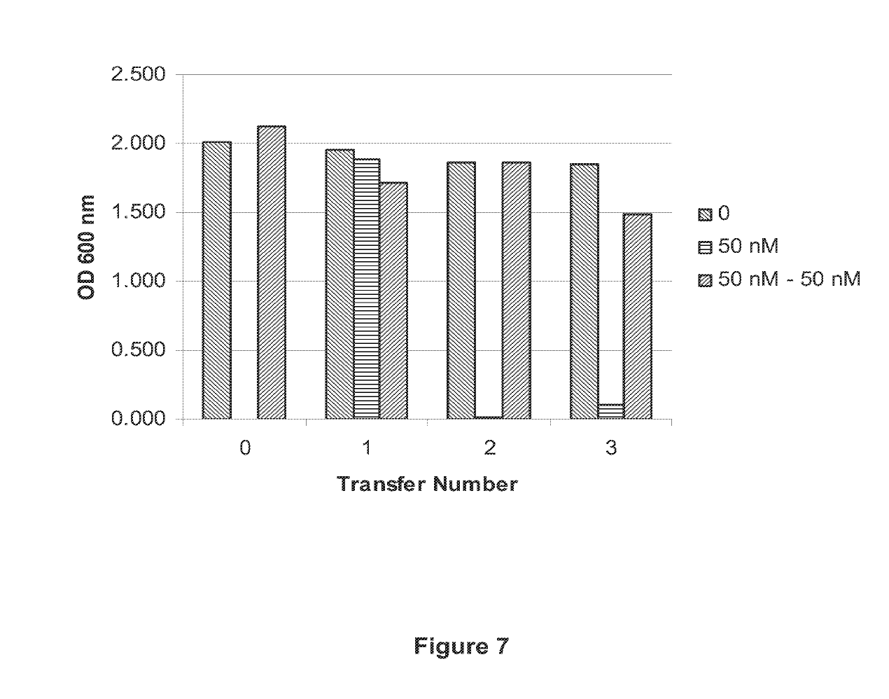

Effect of peptide SEQ ID NO:143 addition on sequential batch cultures of C. acetobutylicum ATCC 824 grown at 37° C. Spores of C. acetobutylicum ATCC 824 were germinated and grown overnight at 37° C. under anaerobic conditions in YEPG medium that either contained 50 nM of peptide SEQ ID NO:143 or no added peptide. After about 24 h of growth, 10 μL of the untreated culture was transferred (transfer 1) to each of two flasks that contained 10 mL of YEPG with either no treatment or with 50 nM peptide SEQ ID NO:143. At the same time, 10 μL of the culture that was germinated in the presence of peptide SEQ ID NO:143 was also transferred to 10 mL of YEPG that contained 50 nM of peptide SEQ ID NO:143. Thereafter, 10 μL of each culture was transferred, at the same time, every 24-48 h to 10 mL of fresh YEPG that contained the same peptide treatment or no treatment. Each culture was stopped after 72 hours if incubation and optical density, pH and ceric ion reactive chemicals were measured. Sequential batch culturing was continued through 3 transfers at which point the untreated culture and the culture that was germinated and transferred in 50 nM of peptide were still growing, while the culture that was treated with peptide after germination had stopped growing (Table 15 and FIG. 7).

TABLE 15
Optical density at 600 nm of C. acetobutylicum ATCC 824 72
h culture broths following germination and sequential transfers
in the absence and presence of peptide SEQ ID NO: 143.

| | Peptide Concentrations (nM) | | |
|---|---|---|---|
| Transfer | 0 | 50 | 50-50^{a} |
| 0^{b} | 2.010 | | 2.121 |
| 1 | 1.954 | 1.891 | 1.715 |
| 2 | 1.869 | 0.011 | 1.858 |
| 3 | 1.848 | 0.100 | 1.485 |

^{a}C. acetobutylicum spores were germinated in the presence of 50 nM peptide SEQ ID: NO 143.
^{b}The cultures of germinated C. acetobutylicum spores were not considered culture transfers.

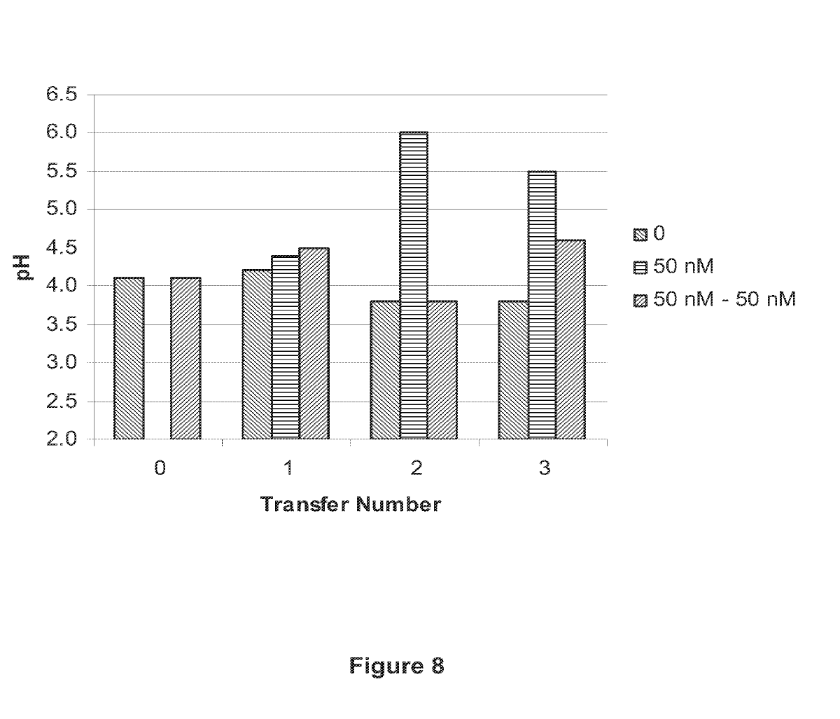

The final pH of the culture that was treated with peptide after germination was similar to the other two cultures at the first transfer, but then rose to pH 6.0 with no apparent growth and then decreased to pH 5.5 at the third transfer with a slight amount of growth (Table 16 and FIG. 8). The decrease of culture pH and slight increase in optical density (see Table 15, above) suggested that the growth of this culture was inhibited but it was still metabolically active. Final pH of the other two cultures remained similar through the three transfers, although, pH of the culture that had been germinated in the presence of peptide SEQ ID NO:143 was higher than that of the untreated culture at the third transfer.

TABLE 16
Final pH of C. acetobutylicum ATCC 824 72 h culture
broths following germination and sequential transfers
in the absence and presence of peptide SEQ ID NO: 143.

| | Peptide Concentrations (nM) | | |
|---|---|---|---|
| Transfer | 0 | 50 | 50-50^{a} |
| 0^{b} | 4.1 | | 4.1 |
| 1 | 4.2 | 4.4 | 4.5 |
| 2 | 3.8 | 6.0 | 3.8 |
| 3 | 3.8 | 5.5 | 4.6 |

^{a}C. acetobutylicum spores were germinated in the presence of 50 nM peptide SEQ ID NO: 143.
^{b}The cultures of germinated C. acetobutylicum spores were not considered culture transfers.

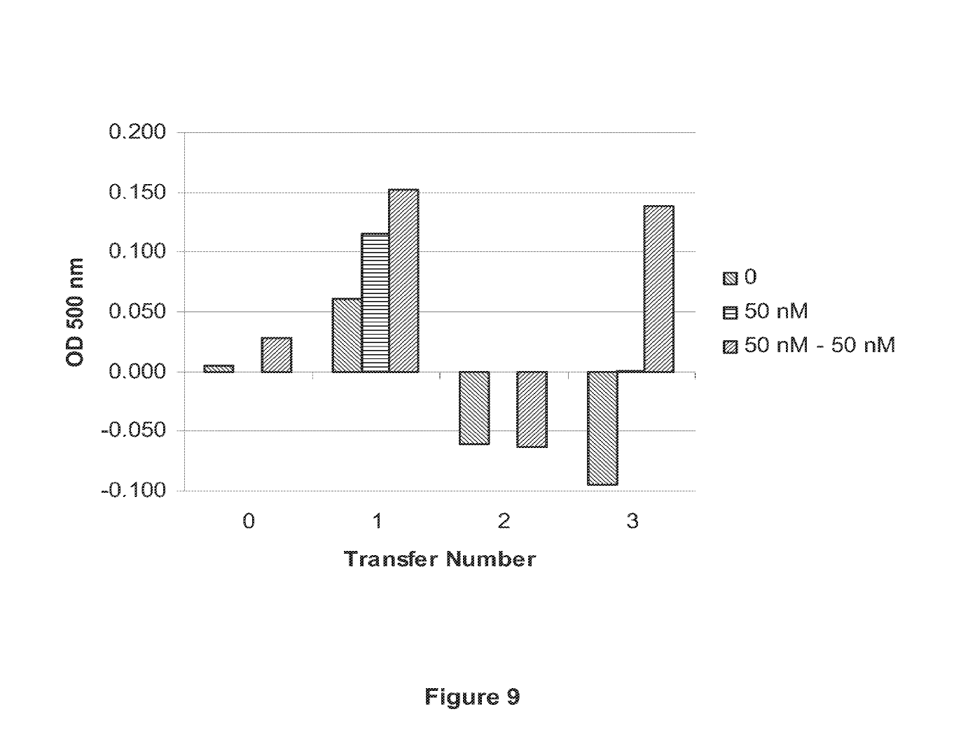

The presence of ceric ion reactive chemicals was also affected by the addition of peptide SEQ ID NO:143 during germination and subsequent sequential batch cultures at 37° C. (Table 17 and FIG. 9). At the first transfer, all cultures were positive for ceric ion reactive compounds, although, both peptide treated cultures had higher measurements than the untreated culture. Both growing cultures (see Table 15) had optical density readings less than zero at the second transfer, and the untreated culture continued to decline at the third transfer while the culture that had been germinated and grown in the presence of peptide SEQ ID NO:143 returned to a positive value.

TABLE 17
Optical density of ceric ion reactive compounds measured
at 500 nm in C. acetobutylicum ATCC 824 72 h culture
broths following germination and sequential transfers
in the absence and presence of peptide SEQ ID NO: 143.

| | Peptide Concentrations (nM) | | |
|---|---|---|---|
| Transfer | 0 | 50 | 50-50^{a} |
| 0^{b} | 0.005 | | 0.028 |
| 1 | 0.061 | 0.116 | 0.152 |
| 2 | −0.061 | 0.000 | −0.063 |
| 3 | −0.095 | 0.001 | 0.138 |

^{a}C. acetobutylicum spores were germinated in the presence of 50 nM peptide SEQ ID NO: 143.
^{b}The cultures of germinated C. acetobutylicum spores were not considered culture transfers.

Peptide treated cultures responded differently at 37° C. than at 30° C. At 37° C., the untreated culture survived through 3 transfers while the treated culture did not grow beyond the first transfer. However, when the culture that was germinated in 50 nM of peptide SEQ ID NO:143 and then transferred with peptide treatment, the culture continued through the third transfer, although to a slightly lower final value at 72 h compared to the untreated culture. Also, while ceric ion reactive compounds produced by the untreated culture decreased steadily from the first through third transfer, the culture that was germinated and transferred with peptide treatment oscillated from a high value at the first transfer to a lower value at the second and back to a high value at the third transfer. At 37° C., peptide treatment during germination and growth prevented culture degeneration in terms of production of total alcohols.

### Example 9

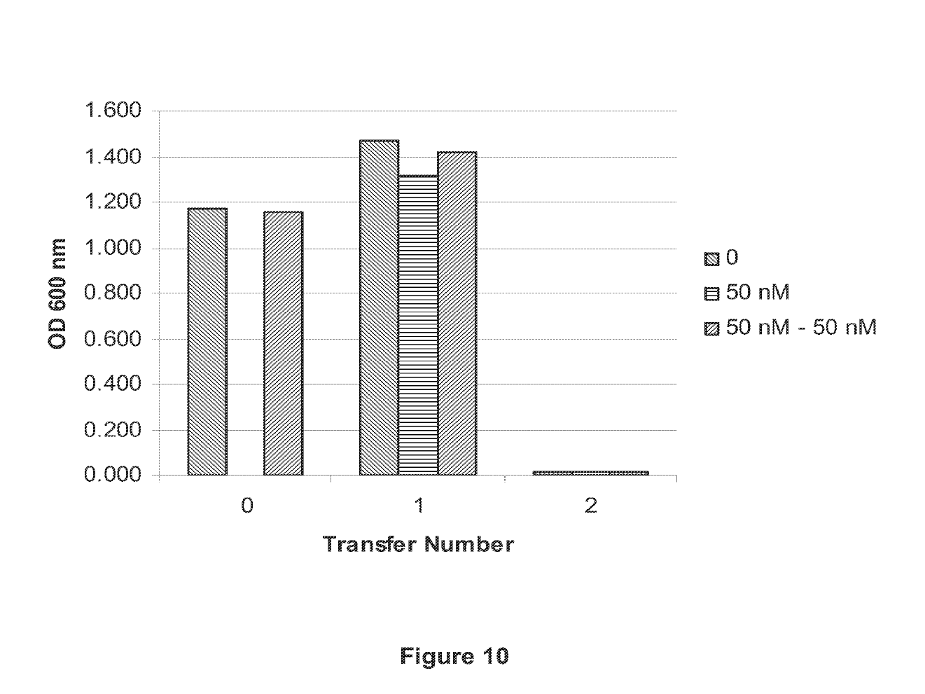

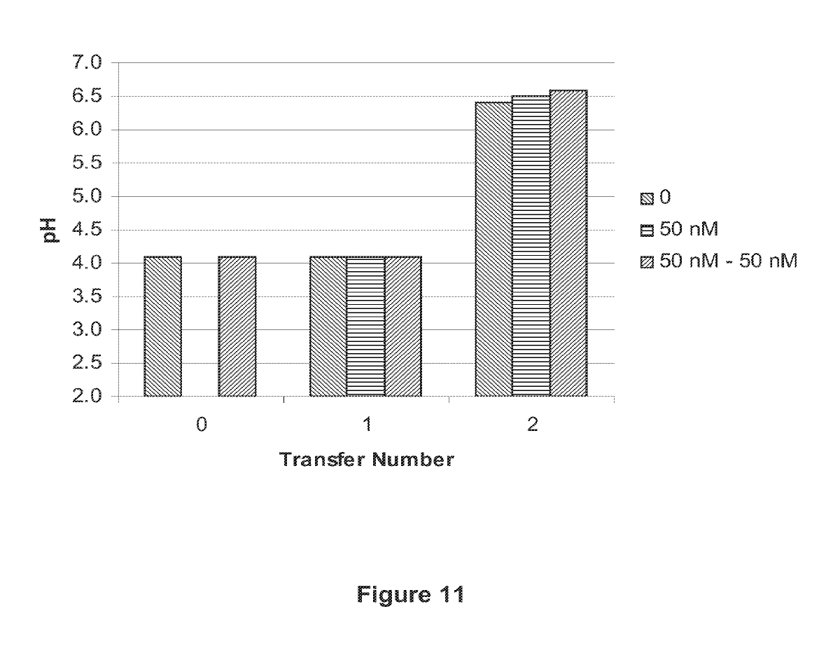

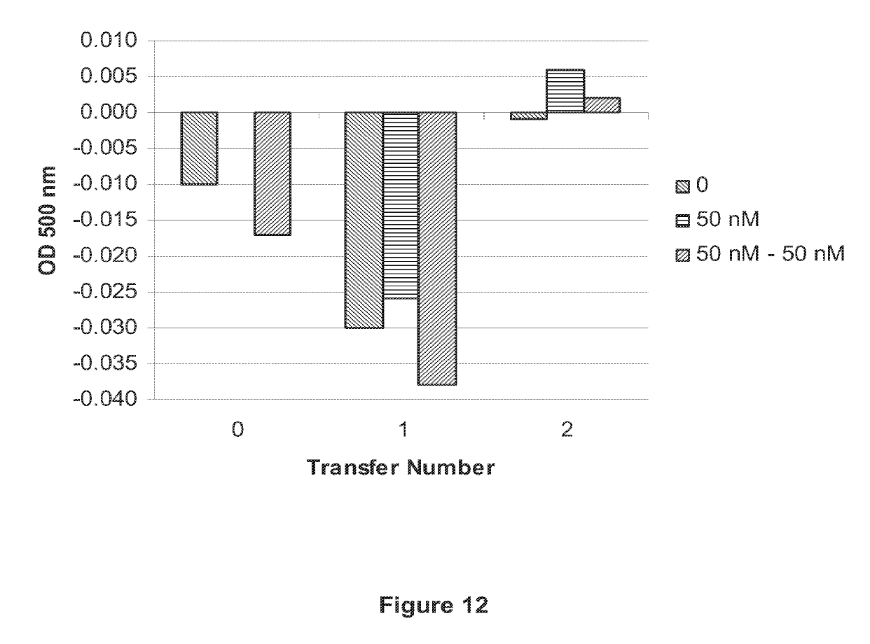

Effect of peptide SEQ ID NO:145 addition on sequential batch cultures of C. beijerinckii NCIMB 8052 grown at 37° C. Spores of C. beijerinckii NCIMB 8052 were germinated and grown overnight at 37° C. under anaerobic conditions in YEPG medium that either contained 50 nM of peptide SEQ ID NO:145 or no added peptide. After about 24 h of growth, 10 μL of the untreated culture was transferred (transfer 1) to each of two flasks that contained 10 mL of YEPG with either no treatment or with 50 nM peptide SEQ ID NO:145. At the same time, 10 μL of the culture that was germinated in the presence of peptide SEQ ID NO:145 was also transferred to 10 mL of YEPG that contained 50 nM of peptide SEQ ID NO:145. Thereafter, 10 μL of each culture was transferred, at the same time, every 24-48 h to 10 mL of fresh YEPG that contained the same peptide treatment or no treatment. Each culture was stopped after 72 hours of incubation and optical density, pH and ceric ion reactive chemicals were measured. Addition of peptide SEQ ID NO:145 appeared to have no effect on endpoint measurements of the growth of C. beijerinckii NCIMB 8052 after germination or during sequential transfers of cultures at 37° C. (Table 18 and FIG. 10). All three cultures stopped growing at the second transfer. Likewise, there was no apparent effect on endpoint measurements of pH or ceric ion reactive compounds (Tables 19 and 20 and FIGS. 11 and 12).

TABLE 18
Optical density at 600 nm of C. beijerinckii NCIMB 8052 72
h culture broths following germination and sequential transfers
in the absence and presence of peptide SEQ ID NO: 145.

| | Peptide Concentrations (nM) | | |
|---|---|---|---|
| Transfer | 0 | 50 | 50-50^{a} |
| 0^{b} | 1.172 | | 1.158 |
| 1 | 1.472 | 1.313 | 1.420 |
| 2 | 0.012 | 0.011 | 0.011 |

^{a}C. beijerinckii spores were germinated in the presence of 50 nM peptide SEQ ID NO: 145.
^{b}The cultures of germinated C. beijerinckii spores were not considered culture transfers.

TABLE 19
Final pH of C. beijerinckii NCIMB 8052 72 h culture
broths following germination and sequential transfers
in the absence and presence of peptide SEQ ID NO: 145.

| | Peptide Concentrations (nM) | | |
|---|---|---|---|
| Transfer | 0 | 50 | 50-50^{a} |
| 0^{b} | 4.1 | | 4.1 |
| 1 | 4.1 | 4.1 | 4.1 |
| 2 | 6.4 | 6.5 | 6.6 |

^{a}C. beijerinckii spores were germinated in the presence of 50 nM peptide SEQ ID NO: 145.
^{b}The cultures of germinated C. beijerinckii spores were not considered culture transfers.

TABLE 20
Optical density of ceric ion reactive compounds measured
at 500 nm in C. beijerinckii NCIMB 8052 72 h culture
broths following germination and sequential transfers
in the absence and presence of peptide SEQ ID NO: 145.

| | Peptide Concentrations (nM) | | |
|---|---|---|---|
| Transfer | 0 | 50 | 50-50^{a} |
| 0^{b} | −0.010 | | −0.017 |
| 1 | −0.030 | −0.026 | −0.038 |
| 2 | −0.001 | 0.006 | 0.002 |

^{a}C. beijerinckii spores were germinated in the presence of 50 nM peptide SEQ ID NO: 145.
^{b}The cultures of germinated C. beijerinckii spores were not considered culture transfers.

Although the endpoint data for C. beijerinckii NCIMB 8052 grown at 37° C. look identical at transfer 1, regardless of treatment, visual observations through the course of growth indicated that the untreated culture grew first whereas the treated culture grew later. Peptide SEQ ID NO:145, therefore, had a repressive effect on germination and growth of C. beijerinckii NCIMB 8052 when grown at 37° C. The gene sequence that encodes peptide SEQ ID NO: 145 is a potential candidate for genetic modification to reduce or eliminate formation of the peptide, which should reduce or eliminate the antagonistic effect on growth and butanol formation.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

1-52. (canceled)

53. A composition comprising, a recombinant, or chemically synthesized peptide whereby the peptide binds to one or more quorum sensing regulatory proteins of Clostridium acetobutylicum, and modifies the differentiated state of the Clostridium acetobutylicum in culture.

54. The composition of claim 53, whereby one or more quorum sensing regulatory proteins are selected from the group consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62,

55. The composition of claim 53, whereby the recombinant or chemically synthesized peptide consists of an amino acid sequence at lest 90 percent identical to a sequence in selected from the group consisting of SEQ ID NO:143, SEQ ID NO:144, SEQ ID NO:145, SEQ ID NO:146, SEQ ID NO:147, and SEQ ID NO:148.

56. The composition of claim 53, whereby the recombinant or chemically synthesized peptide consists of an amino acid sequence set forth in SEQ ID NO:143, and conservatively substituted variants thereof.

57. (canceled)

58. The composition of claim 53, whereby the recombinant or chemically synthesized peptide consists of an amino acid sequence set forth in SEQ ID NO:143.

59. The composition of claim 53, whereby the recombinant or chemically synthesized peptide consists of an amino acid sequence set forth in SEQ ID NO:144, and conservatively substituted variants thereof.

60. The composition of claim 53, whereby the recombinant or chemically synthesized peptide consists of a peptide with an amino acid sequence set forth in SEQ ID NO:144.

61. The composition of claim 53, whereby the recombinant or chemical synthesized peptide consists of an amino acid sequence at lest 90 percent identical to the sequence set forth in SEQ ID NO: 146.

62. (canceled)

63. The composition of claim 53, further comprising media capable of sustaining Clostridium acetobutylicum in culture.

64. The composition of claim 53, whereby the amount of peptide is equal to an effective amount.

65. The composition of claim 53, whereby the Clostridium acetobutylicum consists of Clostridium acetobutylicum ATCC 824.

66. The composition of claim 53, whereby the differentiated state is extended serial propagation.

67. A method for directing or maintaining the differentiated state of a culture of Clostridium acetobutylicum comprising, culturing the Clostridium acetobutylicum in a media comprising an effective amount of the peptide of claim 53.

68. The method of claim 67, whereby the peptide is 90 percent identical to the sequence selected from the group consisting of SEQ ID NO:143, SEQ ID NO:144, SEQ ID NO:145, SEQ ID NO:146, SEQ ID NO:147, and SEQ ID NO:148.

69. A method for directing or maintaining the differentiated state of Clostridium acetobutylicum in culture, comprising modifying the activity of one or more quorum sensing regulatory proteins.

70. The method of claim 69 whereby one or more quorum sensing regulatory proteins are selected from the group consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62.

71. The method of claim 69 whereby modifying the activity of one or more quorum sensing regulatory proteins comprises reducing or eliminating the activity through genetic engineering of the Clostridium acetobutylicum.

72. (canceled)

73. The method of claim 67, whereby the peptide consisting of SEQ ID NO:143, and conservatively substituted variants thereof.

74. (canceled)

75. (canceled)
